SEQUENCE LISTING (166 sequences)

SEQ ID NO: 1 - RNA, 21 nt, Medicago truncatula
ugaacagaaagaacauuuggc

SEQ ID NO: 2 - RNA, 21 nt, Medicago truncatula
uagggacuaaauugaugguuu

SEQ ID NO: 3 - RNA, 21 nt, Medicago truncatula
auucaagaaggucguggaaaa

SEQ ID NO: 4 - RNA, 21 nt, Medicago truncatula
ccacaucaauggucguuggau

SEQ ID NO: 5 - RNA, 22 nt, Medicago truncatula
ugagaacuguuuuccgcaccuu

SEQ ID NO: 6 - RNA, 22 nt, Medicago truncatula
uagguauuuuaaggagcacguu

SEQ ID NO: 7 - RNA, 24 nt, Medicago truncatula
cgugacaacacaucauuggaugca

SEQ ID NO: 8 - RNA, 24 nt, Medicago truncatula
uggauucaacucuaagugucgguu

SEQ ID NO: 9 - RNA, 24 nt, Medicago truncatula
aaaagaauacucauacauaacauu

SEQ ID NO: 10 - RNA, 21 nt, Medicago truncatula
acaaagacauuggaaggcuua

SEQ ID NO: 11 - RNA, 22 nt, Medicago truncatula
uucggguugauaauauuuucuu

SEQ ID NO: 12 - RNA, 21 nt, Medicago truncatula
ugugaaugaugcgggagcuaa

SEQ ID NO: 13 - RNA, 22 nt, Medicago truncatula
uaaauguguuggaauuaagguu

SEQ ID NO: 14 - RNA, 22 nt, Medicago truncatula
ugauagagcuagaccaucggag

SEQ ID NO: 15 - RNA, 24 nt, Medicago truncatula
aagagagauagaucagaauugaca

SEQ ID NO: 16 - RNA, 21 nt, Medicago truncatula
uugauuugaucagaugguuuu

SEQ ID NO: 17 - RNA, 20 nt, Medicago truncatula
uugaucaaggacuuugcauc

SEQ ID NO: 18 - RNA, 21 nt, Medicago truncatula
cauuguuugguuuaguacaaa

SEQ ID NO: 19 - RNA, 21 nt, Medicago truncatula
auuuugauuggugccaacuaa

SEQ ID NO: 20 - RNA, 22 nt, Medicago truncatula
uauugcacucaucuuccauggc

SEQ ID NO: 21 - RNA, 21 nt, Medicago truncatula
cuuuaacgcgggaaagacaca

SEQ ID NO: 22 - RNA, 21 nt, Medicago truncatula
ugaugacggaagaaauccaaa

SEQ ID NO: 23 - RNA, 21 nt, Medicago truncatula
ucugucaguagacucaauuuc

SEQ ID NO: 24 - RNA, 21 nt, Medicago truncatula
ugaaaguguagagaguagauu

SEQ ID NO: 25 - RNA, 21 nt, Medicago truncatula
ugaaaguguggagaguagauu

SEQ ID NO: 26 - RNA, 21 nt, Medicago truncatula
uuagcaggaagagugacuaug

SEQ ID NO: 27 - RNA, 21 nt, Medicago truncatula
uuuuccaauucuaagucuauc

SEQ ID NO: 28 - RNA, 21 nt, Medicago truncatula
ccgaccggauucucagacggg

SEQ ID NO: 29 - RNA, 21 nt, Medicago truncatula
uagagauuuguuuaacagcca

SEQ ID NO: 30 - RNA, 21 nt, Medicago truncatula
ugagaauguuagauacggaac

SEQ ID NO: 31 - RNA, 21 nt, Medicago truncatula
uacuuggugaauuguuggauc

SEQ ID NO: 32 - RNA, 21 nt, Medicago truncatula
uaaguuggaagaauguauuug

SEQ ID NO: 33 - RNA, 21 nt, Medicago truncatula
uuaugcaaguagauaagcuca

SEQ ID NO: 34 - RNA, 21 nt, Medicago truncatula
uuuaauuuauauacaucguca

SEQ ID NO: 35 - RNA, 21 nt, Medicago truncatula
aagggggugaaaagauucaaa

SEQ ID NO: 36 - RNA, 24 nt, Medicago truncatula
auacaucauugauugaaugaaccu

SEQ ID NO: 37 - RNA, 24 nt, Medicago truncatula
ugcuuuaaggauauuuguuaagga

SEQ ID NO: 38 - RNA, 23 nt, Medicago truncatula
augggauccuguugguggguuac

SEQ ID NO: 39 - RNA, 21 nt, Medicago truncatula
aacccucaaaggcuuccacgg

SEQ ID NO: 40 - RNA, 22 nt, Medicago truncatula
gugggauccguugauggguuac

SEQ ID NO: 41 - RNA, 21 nt, Medicago truncatula
cauuuggagagacauagacaa

SEQ ID NO: 42 - RNA, 21 nt, Medicago truncatula
ugggagaaaagauagaaugug

SEQ ID NO: 43 - RNA, 22 nt, Glycine max
ggagaugggagggucgguaaag

SEQ ID NO: 44 - RNA, 21 nt, Glycine max
uuuugugucgugaagcuuuug

SEQ ID NO: 45 - RNA, 21 nt, Glycine max
cauggaagugaaucgggugac

SEQ ID NO: 46 - RNA, 21 nt, Glycine max
ucauaggaggaaucaacuggc

SEQ ID NO: 47 - RNA, 21 nt, Glycine max
ugaacuauacaaagacgguua

SEQ ID NO: 48 - RNA, 22 nt, Glycine max
uccauucgcggacaugauggau

SEQ ID NO: 49 - RNA, 24 nt, Glycine max
cgaaacguugagguauauguggac

SEQ ID NO: 50 - RNA, 21 nt, Glycine max
uugaggcugagaagaggcaag

SEQ ID NO: 51 - RNA, 21 nt, Glycine max
uggaggaccuuugaaggugca

SEQ ID NO: 52 - RNA, 24 nt, Glycine max
aagugcaauacugaucuucggaac

SEQ ID NO: 53 - RNA, 21 nt, Glycine max
ugagggaaaugaagacgacga

SEQ ID NO: 54 - RNA, 21 nt, Glycine max
uuaggacuaugguuuggacga

SEQ ID NO: 55 - RNA, 22 nt, Glycine max
ccugucguaggagagaugacgc

SEQ ID NO: 56 - RNA, 21 nt, Glycine max
uuugggaucaugcuaucccuu

SEQ ID NO: 57 - RNA, 24 nt, Glycine max
aucucaaguggauugcuuaaggac

SEQ ID NO: 58 - RNA, 21 nt, Glycine max
cugggugagagaaacacguau

SEQ ID NO: 59 - RNA, 24 nt, Glycine max
agaaugugaguuagagugagcauc

SEQ ID NO: 60 - RNA, 22 nt, Glycine max
uuuuuaaaagguucaguuaggu

SEQ ID NO: 61 - RNA, 21 nt, Glycine max
gcuggcgucgacacguggcau

SEQ ID NO: 62 - RNA, 21 nt, Glycine max
ugggugagagaaacgcguauc

SEQ ID NO: 63 - RNA, 24 nt, Glycine max
aaugucugggcuuagcgaggcggu

SEQ ID NO: 64 - RNA, 21 nt, Glycine max
auaagcucuuuugagagcuuc

SEQ ID NO: 65 - RNA, 21 nt, Glycine max
aaagccaugacuuacacacgc

SEQ ID NO: 66 - RNA, 21 nt, Glycine max
uuauagucugacaucuggaau

SEQ ID NO: 67 - RNA, 21 nt, Glycine max
aacuugggcugagcuuaggug

SEQ ID NO: 68 - RNA, 22 nt, Glycine max
cuagcaauaauguuggaugcac

SEQ ID NO: 69 - RNA, 22 nt, Glycine max
cgacgaacucuucgucggcauc

SEQ ID NO: 70 - RNA, 24 nt, Glycine max
caaguccaaaguaggaauguugca

SEQ ID NO: 71 - RNA, 23 nt, Glycine max
aucacuuagcugacgguagggac

SEQ ID NO: 72 - RNA, 22 nt, Glycine max
cugaaacugagacugcaucugg

SEQ ID NO: 73 - RNA, 21 nt, Glycine max
uagcugguaggagaaguucag

SEQ ID NO: 74 - RNA, 22 nt, Glycine max
gacgacgacggggaggacgcgc

SEQ ID NO: 75 - RNA, 21 nt, Glycine max
aaacagaucuaaauggauucc

SEQ ID NO: 76 - RNA, 21 nt, Glycine max
uuaggacuaugguuuggacaa

SEQ ID NO: 77 - RNA, 21 nt, Glycine max
aauuagcuaaugguuagcuaa

SEQ ID NO: 78 - RNA, 24 nt, Glycine max
uaguguuguccugucgaacacgga

SEQ ID NO: 79 - RNA, 20 nt, Glycine max
aguggaacuuugaggccugc

SEQ ID NO: 80 - RNA, 21 nt, Glycine max
ugagaauuuggccucugucca

SEQ ID NO: 81 - RNA, 21 nt, Glycine max
uaauuguguuguacauuauca

SEQ ID NO: 82 - RNA, 22 nt, Medicago truncatula
uugacagaagagggugagcaca

SEQ ID NO: 83 - RNA, 21 nt, Medicago truncatula
acagggaacaugcagagcaug

SEQ ID NO: 84 - RNA, 21 nt, Medicago truncatula
agcugcugacucguugguuca

SEQ ID NO: 85 - RNA, 22 nt, Medicago truncatula
ggcaggucauccuucggcuaua

SEQ ID NO: 86 - RNA, 22 nt, Medicago truncatula
uacgugugucuucaccucugaa

SEQ ID NO: 87 - RNA, 21 nt, Medicago truncatula
cgagccgaaucaauaucacuc

SEQ ID NO: 88 - RNA, 21 nt, Medicago truncatula
ucucggaccaggcuucauucc

SEQ ID NO: 89 - RNA, 21 nt, Medicago truncatula
uugagccgcgccaauaucacu

SEQ ID NO: 90 - RNA, 21 nt, Medicago truncatula
gcucaagaaagcugugggaga

SEQ ID NO: 91 - RNA, 22 nt, Medicago truncatula
ugagccaaagaugacuugccgg

SEQ ID NO: 92 - RNA, 21 nt, Medicago truncatula
ucgcuuggugcaggucgggaa

SEQ ID NO: 93 - RNA, 21 nt, Medicago truncatula
aucaugcuaucccuuuggauu

SEQ ID NO: 94 - RNA, 22 nt, Medicago truncatula
ggagugacacugagaacacaag

SEQ ID NO: 95 - RNA, 22 nt, Medicago truncatula
ucgcaggagagaugacaccuuc

SEQ ID NO: 96 - RNA, 22 nt, Medicago truncatula
uuugguacuucgucgauuugau

SEQ ID NO: 97 - RNA, 22 nt, Medicago truncatula
uuaaucuaggaaaauacggugg

SEQ ID NO: 98 - RNA, 21 nt, Medicago truncatula
ugaagcugccagcaugaucug

SEQ ID NO: 99 - RNA, 21 nt, Medicago truncatula
auccuuggaaugcagauuauc

SEQ ID NO: 100 - RNA, 21 nt, Medicago truncatula
uuuggcauucuguccaccucc

SEQ ID NO: 101 - RNA, 21 nt, Medicago truncatula
agaaucuugaugaugcugcau

SEQ ID NO: 102 - RNA, 21 nt, Medicago truncatula
auuggagugaagggagcucca

SEQ ID NO: 103 - RNA, 21 nt, Medicago truncatula
ugcauuugcaccugcacuuuc

SEQ ID NO: 104 - RNA, 21 nt, Glycine max
ugccaaaggagaguugcccug

SEQ ID NO: 105 - RNA, 21 nt, Glycine max
caggggaacaggcagagcaug

SEQ ID NO: 106 - RNA, 21 nt, Glycine max
gcguaugaggagccaagcaua

SEQ ID NO: 107 - RNA, 21 nt, Glycine max
uauggggggauugggaaggaa

SEQ ID NO: 108 - RNA, 21 nt, Glycine max
uuggacugaagggagcucccu

SEQ ID NO: 109 - RNA, 22 nt, Glycine max
ucuugcucaaaugaguauucca

SEQ ID NO: 110 - RNA, 21 nt, Glycine max
uugacagaagaaagggagcac

SEQ ID NO: 111 - RNA, 21 nt, Glycine max
ugcauuugcaccugcacuuua

SEQ ID NO: 112 - RNA, 21 nt, Glycine max
uugagccgcgucaauaucuca

SEQ ID NO: 113 - RNA, 21 nt, Glycine max
ugaaucuugaugaugcugcau

SEQ ID NO: 114 - RNA, 21 nt, Glycine max
ugacagaagagagugagcacu

SEQ ID NO: 115 - RNA, 21 nt, Glycine max
uugagccgcgccaauaucacu

SEQ ID NO: 116 - RNA, 21 nt, Glycine max
gcucaagaaagcugugggaga

SEQ ID NO: 117 - RNA, 21 nt, Glycine max
uugagccgugccaauaucaca

SEQ ID NO: 118 - RNA, 21 nt, Glycine max
uacgcaggagagaugacgcug

SEQ ID NO: 119 - RNA, 21 nt, Glycine max
ggaaucuugaugaugcugcau

SEQ ID NO: 120 - RNA, 23 nt, Glycine max
ugagccgggauggcuugccggca

SEQ ID NO: 121 - RNA, 22 nt, Glycine max
ugagccaggauggcuugccggc

SEQ ID NO: 122 - RNA, 21 nt, Glycine max
uuuggaccgaagggagccccu

SEQ ID NO: 123 - RNA, 21 nt, Glycine max
uauugacgcugcacucaauca

SEQ ID NO: 124 - RNA, 22 nt, Glycine max
cgggcaaguuguuuuuggcuac

SEQ ID NO: 125 - RNA, 21 nt, Glycine max
aggugggcauacugucaacug

SEQ ID NO: 126 - RNA, 20 nt, Glycine max
uuggcauucuguccaccucc

SEQ ID NO: 127 - RNA, 21 nt, Glycine max
ugagaccaaaugagcagcuga

SEQ ID NO: 128 - RNA, 21 nt, Glycine max
uagaagcuccccauguucuca

SEQ ID NO: 129 - RNA, 21 nt, Glycine max
ugagccaaggaugacuugccg

SEQ ID NO: 130 - RNA, 21 nt, Glycine max
ucucggaccaggcuucauucc

SEQ ID NO: 131 - RNA, 21 nt, Glycine max
cugacagaagauagagagcau

SEQ ID NO: 132 - RNA, 21 nt, Glycine max
agggauagguaaaacaaugac

SEQ ID NO: 133 - RNA, 21 nt, Glycine max
uagaaagggaaauagcaguug

SEQ ID NO: 134 - RNA, 21 nt, Glycine max
ugauugagccgugccaauauc

SEQ ID NO: 135 - RNA, 21 nt, Glycine max
auucaagauagcuguggaaaa

SEQ ID NO: 136 - RNA, 21 nt, Glycine max
ucgauaaaccucugcauccag

SEQ ID NO: 137 - RNA, 21 nt, Glycine max
agcucuguuggcuacacuuug

SEQ ID NO: 138 - RNA, 21 nt, Glycine max
aagccaaggaugacuugccgg

SEQ ID NO: 139 - RNA, 21 nt, Glycine max
aaccaggcucugauaccaugg

SEQ ID NO: 140 - RNA, 24 nt, Glycine max
aaguugugaugagaaucaauggca

SEQ ID NO: 141 - RNA, 21 nt, Glycine max
uaaucugcauccugagguuua

SEQ ID NO: 142 - RNA, 21 nt, Glycine max
cugacagaagauagagagcac

SEQ ID NO: 143 - RNA, 21 nt, Glycine max
ugucgcaggauagagggcacu

SEQ ID NO: 144 - RNA, 21 nt, Glycine max
augcacugccucuucccuggc

SEQ ID NO: 145 - RNA, 21 nt, Glycine max
guccuugggaugcagauuacg

SEQ ID NO: 146 - RNA, 21 nt, Glycine max
aaaagcacuuaaggaacggua

SEQ ID NO: 147 - RNA, 21 nt, Glycine max
agggauagguaaaacaacuac

SEQ ID NO: 148 - RNA, 21 nt, Glycine max
ggaaugggcugauugggaagc

SEQ ID NO: 149 - RNA, 22 nt, Glycine max
uaacugaacauucuuagagcau

SEQ ID NO: 150 - RNA, 22 nt, Glycine max
uaacuggaaauucuuaaagcau

SEQ ID NO: 151 - RNA, 21 nt, Glycine max
uagccaagaaugacuugccgg

SEQ ID NO: 152 - RNA, 21 nt, Glycine max
cugggaacaggcagggcacga

SEQ ID NO: 153 - RNA, 21 nt, Glycine max
cgagccgaaucaauaccacuc

SEQ ID NO: 154 - RNA, 21 nt, Glycine max
ugcgagugucuucgccucuga

SEQ ID NO: 155 - RNA, 21 nt, Glycine max
ugcggguaucuuugccucuga

SEQ ID NO: 156 - RNA, 22 nt, Glycine max
uuaaucaaggaaaucacgguug

SEQ ID NO: 157 - RNA, 21 nt, Glycine max
ugccaaaggagauuugcccag

SEQ ID NO: 158 - RNA, 22 nt, Glycine max
uagccaaggauggacuugccua

SEQ ID NO: 159 - RNA, 21 nt, Glycine max
ggaauguugucuggcucgagg

SEQ ID NO: 160 - RNA, 21 nt, Glycine max
cuuccauaucuggggagcuuc

SEQ ID NO: 161 - RNA, 21 nt, Glycine max
auugagugcagcguugaugaa

SEQ ID NO: 162 - RNA, 21 nt, Glycine max
uguguucucaggucgccccug

SEQ ID NO: 163 - RNA, 21 nt, Glycine max
ugaaguguuugggggaacuuu

SEQ ID NO: 164 - RNA, 20 nt, Glycine max
cggaccaggcuucauucccc

SEQ ID NO: 165 - RNA, 21 nt, Glycine max
agagguguuugggaugagaga

SEQ ID NO: 166 - RNA, 21 nt, Glycine max
cagccaaggaugacuugccgu

(19) United States
(12) Patent Application Publication
(10) Pub. No.: US 2014/0356961 A1
(43) Pub. Date: Dec. 4, 2014

(54) USE OF PHASED SMALL RNAS FOR SUPPRESSION OF PLANT DEFENSE AND OTHER PLANT GENES

(71) Applicants: Blake Meyers, Wilmington, DE (US); Jixian Zhai, Newark, DE (US); Janine Sherrier, Hockessin, DE (US)

(72) Inventors: Blake Meyers, Wilmington, DE (US); Jixian Zhai, Newark, DE (US); Janine Sherrier, Hockessin, DE (US)

(73) Assignee: University of Delaware Technology Park, Newark, DE (US)

(21) Appl. No.: 14/234,174

(22) PCT Filed: Jul. 20, 2012
(86) PCT No.: PCT/US12/47627

§ 371 Date: Aug. 21, 2014

Related U.S. Application Data
(60) Provisional application No. 61/510,744, filed on Jul. 22, 2011.

(51) Int. Cl. C12N 15/82

(52) U.S. Cl. CPC C12N 15/8218
USPC 435/468

(57) ABSTRACT

A method for generating a phased siRNA (phasiRNA) in a plant cell comprises introducing into the plant cell an effective amount of a microRNA (miRNA). A transcript of a NB-LRR encoding gene may be cleaved in the plant cell. Resistance of the plant to a microbe may be reduced. The plant may enter a symbiotic interaction with a microbe. Nodulation in the plant may be improved. Related compositions are provided.

### CROSS-REFERENCE TO RELATED APPLICATION

This application claims the benefit of U.S. Provisional Application No. 61/510,744, filed Jul. 22, 2011, the contents of which are incorporated herein in their entireties for all purposes.

### GOVERNMENT INTERESTS

This invention was made with government support under Grant No. 2006-03567 awarded by the United States Department of Agriculture (USDA). The government has certain rights in this invention.

### FIELD OF THE INVENTION

The invention relates generally to microRNAs and the use thereof for suppression of plant defense and other plant genes as well as triggering production of phased small RNAs in plant cells.

### BACKGROUND OF THE INVENTION

Legume species are agronomically important crops that are rich sources of human dietary protein that also develop unique nitrogen-fixing nodules through a symbiotic relationship with microbes. Root nodules house symbiotic bacteria (rhizobia) that convert atmospheric di-nitrogen to ammonia using the energy of the host's photosynthate. During establishment of the symbiosis, rhizobia invade the legume root via a plant-derived infection thread and are ultimately released into the host cytoplasm as membrane-bound facultative organelles called “bacteriods”, which are the differentiated nitrogen-fixing form or rhizobia. While beneficial interactions with mycorrhiza are known for most plant species (with exceptions that include Arabidopsis), the symbiotic relationships that legume species have with bacteria are highly evolved and require many components related to plant pathogenesis. Thus, nodulation may require the suppression of host defenses to prevent immune responses; for example, the classically-defined, allelic Rj2 and Rfg1 loci from soybean restrict nodulation with specific rhizobial strains and encode a TIR-NB-LRR protein. The only known function of plant NB-LRR proteins is in microbial recognition as activators of defense responses. The hundreds of diverse NB-LRRs encoded in plant genomes comprise an innate immune system that allows recognition of many pathogens.

In the last ten years, the functions of small RNAs in plants have been extensively explored. These molecules in their mature form are generally 20 to 24 nucleotides (nt), and are produced by several genetically separable pathways. Plant microRNAs (miRNAs) are typically 21 or 22 nt and function in a post-transcriptional manner by down-regulating target gene products involved in a variety of cellular processes. Another major class of small RNAs are heterochromatic siRNAs (hc-siRNAs) that suppress the activities of transposable elements and maintain genome stability via DNA methylation and chromatin modifications. Trans-acting siRNAs (tasiRNAs) are a third class of plant small RNAs that negatively regulate target transcripts and are characterized by siRNAs spaced in 21-nucleotide “phased” intervals. TasiRNAs have not been extensively described in many plant species. Their formation is dependent on miRNA triggers, and requires either the so-called “two-hit” model of dual miRNA target sites in the non-coding RNA precursor or “one-hit” (single target site) by 22 nt miRNAs. Four families comprising eight tasiRNA loci have been described in Arabidopsis, while hundreds of non-coding loci of unknown function generate phased small RNAs (phasiRNAs) in grasses. The non-coding TAS3 gene is broadly conserved in seed plants.

Legumes and many non-leguminous plants enter symbiotic interactions with microbes, but it remains poorly understood how host plants respond to promote beneficial, symbiotic microbial interactions while suppressing those deleterious or pathogenic. Thus, there remains a need for an effective method to modulate plant defense responses to microbes, especially to promote symbiotic interactions with beneficial microbes.

### SUMMARY OF THE INVENTION

The present invention relates to the use of microRNAs (miRNAs) for triggering production of phased small RNAs (phasiRNAs) in a plant cell, and related compositions.

A method for generating a phased small RNA (phasiRNA) in a plant cell is provided. The method comprises introducing into the plant cell an effective amount of a microRNA (miRNA).

The miRNA may be 22-nt in length. The miRNA may have a sequence selected from the group consisting of SEQ ID NO: 1-166.

The miRNA may be selected from the group consisting of miR156, miR161, miR162, miR167, miR168, miR169, miR172, miR173, miR389*, miR390, miR393, miR472, miR482, miR772, miR828, miR1507, miR1509, miR1510, miR1512, miR1515, miR2089, miR2109, miR2118a, miR2118b, miR2118c, miR2597, miR5300, and miR5754. The miRNA is preferably selected from the group consisting of miR1507 family, miR2109 family, and miR2118 family. The miR1507 family may include gma-miR1507a, mtr-miR1507, gma-miR1507b, vun-miR1507a, vun-miR1507b, gso-miR1507a, gso-miR1507b, and gma-miR1507c. The miR2109 family may include gma-miR2109, and gso-miR2109. The miR2118 family may include miR472, miR482, miR2089, miR2118a, miR2118b, and miR2118c. Examples of the miR2118 family may include pvu-miR2118, mtr-miR2118, osa-miR2118a, osa-miR2118b, osa-miR2118c, osa-miR2118d, osa-miR2118e, osa-miR2118f, osa-miR2118g, osa-miR2118h, osa-miR2118i, osa-miR2118j, osa-miR2118k, osa-miR2118l, osa-miR2118m, osa-miR2118n, osa-miR2118o, osa-miR2118p, osa-miR2118q, osa-miR2118r, zma-miR2118a, zma-miR2118b, zma-miR2118c, zma-miR2118d, zma-miR2118e, zma-miR2118f, zma-miR2118g, gma-miR2118a, gma-miR2118b, vun-miR2118. More preferably, the miRNA is miR482 or miR5300.

A transcript of a NB-LRR encoding gene in the plant cell may be cleaved. The NB-LRR encoding gene may be a disease resistance gene.

The phasiRNA may be generated from a gene selected from group consisting of DCL2 and SGS3.

The plant cell may be in a legume. The legume may be selected from the group consisting of M. truncatula, soybeans, peanuts, and common beans.

The plant cell may be in a non-legume. The non-legume may be selected from the group consisting of corn, rice, wheat, barley, oats, rye, sorghum, sugar cane, grapevine, almonds, apple, peach, sugar beets, tomato, potato, tobacco, cotton, lettuce, sunflower, melons, strawberries, and canola.

Resistance of the plant to a microbe may be reduced. The plant enters a symbiotic interaction with a microbe. The microbe may be a rhizobial strain. Nodulation in the plant may be improved.

### BRIEF DESCRIPTION OF THE DRAWINGS

FIG. 1 shows that twenty-two-nucleotide miRNAs trigger phased siRNA production in M. truncatula. Above are alignments of well-conserved miRNAs and their targets; in the alignment, vertical lines indicate matches, missing lines indicate mismatches, and G:U wobble pairs are indicated with a circle. Black arrowheads above the target sequence indicate the cleavage site in the target, and the numbers above separated by the backslash indicate the number of PARE reads in the small window (W_{S}) (first number) and the number of reads in the large window (W_{L}) (second number), as described in the Zhai 2011 Supplemental Material. Below the alignments are small RNA abundances and phasing score distributions for the regions indicated by the gray trapezoids; abundances are normalized in TPM (transcripts per million). Below this are images from our Web site showing the M. truncatula flower small RNA data at each PHAS locus, with the arrowhead pointing to the miRNA cleavage site. Spots are small RNAs with abundances indicated on the Y-axis indicating primarily either 21-nt or 22-nt secondary sRNAs. Boxes on the bottom or the top strand are annotated exons. Thin, continuous lines of vary distance from the central axis indicate a k-mer frequency for repeats; shading indicates DNA transposons, retrotransposons, or inverted repeats. (A) The new miRNA mtr-miR5754 targets a gene encoding a protein kinase; both the miRNA trigger and the phasiRNAs are specific to the flower library. (8) miR2118 targets a gene encoding a TNL. (C) miR1507 targets a gene encoding a CNL. (D) miR2109 targets a gene encoding a TNL.

FIG. 2 shows novel classes of tasiRNAs identified in the M. truncatula genome. (A) Example of a 2₂₁ TAS locus that encodes an AP2 homolog (Medtr2g093060). The top panel shows the PARE data with a high-abundance tag from the cleaved site (red arrowhead); for space reasons, only the coding strand data are shown for the PARE tags. The image is interpreted as described in FIG. 1. The small RNA data are below; colored dots indicate small RNA sizes, with light blue indicating 21-mers. Other features are as described for the PARE images. The bottom section illustrates the predicted noncleaving miR156 site and the cleaved miR172 site, along with alignments of those miRNAs with the AP2 transcript and the PARE tag abundances. (8) An example of 2₂₂ TAS locus (Medtr7g012810); double cleavage by the 22-nt miRNA miR1509 triggers phasiRNAs. The first cleavage site occurs on Chr. 7 at nucleotide position 3,178,284; the second is at position 3,180,023. Both cleavage sites and alignments are indicated, as in A. (C) The small RNA data from the bottom panel of 8 as a histogram of summed sRNA abundances to emphasize the increased abundance between the two cleavage sites (red arrows in the top panel of 8). The green box outlines the region between the two cleavage sites; the brown box indicates the region 3′ of the 3′ 22-nt miRNA cleavage site to the last gene-associated small RNA.

FIG. 3 shows presence and expression in diverse plant species of 22-nt miRNAs identified from legumes. (Top rows) The presence and abundance of six 22-nt miRNAs that function as phasiRNA triggers were analyzed across 30 species; the intensity of shading indicates the level of expression in each library, according to the key (shown at the bottom of the figure). The abundance is the sum of all variant sequences, allowing up to three mismatches and two nucleotide shifts at either end. The bottom two rows show highly conserved plant miRNAs (miR156 and miR166) in the same libraries as controls for comparison. Each species is indicated by a three-letter code (codes are defined in the legend to Zhai 2011 Supplemental Fig. S6), with two columns for each nonlegume species; the first column is a leaf library, and the second column is a flower library, indicated by “1” or “2” at the bottom of each column. The legume species (“Fabaceae”) are ordered as in Supplemental Table S1. Bars and titles above the species codes indicate the relationships among the species.

FIG. 4 illustrates a model of miRNA triggers and target sites of plant phasiRNA biogenesis. (A) Definition of phased small RNA classes in plants. PhasiRNA-generating loci are called PHAS genes, as tasiRNA-generating loci are TAS genes. (B) Rules and observations of phasiRNAs in plants. The two top cells correspond to PHAS genes with 21-nt miRNA triggers, and the two bottom cells have 22-nt triggers; the left cells have one miRNA-binding site, and the right cells have two binding sites. In each of the four cells, text in boxes is observations of tasiRNAs previously described in Arabidopsis and in this study; lower text in each box indicates which portion of the cleaved TAS transcript is converted to tasiRNAs. In the top left corner of each cell is indicated the name we ascribed to each class; there is no evidence of 1₂₁ phasiRNAs. (C) Examples of phasiRNAs matching the observations described in B.

### DETAILED DESCRIPTION OF THE INVENTION

The present invention is based on the discovery that a few high abundance 22-nt microRNAs (miRNAs) trigger production of phased small RNAs (phasiRNAs) from genes encoding NB-LRR proteins (NB-LRRs). A search for phased siRNAs found at least 114 Medicago loci, the majority of which were defense-related NB-LRR encoding genes (NB-LRRs). Three highly abundant 22-nt miRNA families are identified to target conserved domains in these NB-LRRs and trigger the production of trans-acting siRNAs (tasiRNAs). The only known role of NB-LRRs is in defense, via the detection of pathogen products or the impact of pathogenic attack on plant cells. DCL2 and SGS3 transcripts were also cleaved by these 22-nt miRNAs, generating phasiRNAs, and both of these genes are important for gene silencing and small RNA production. Components of the silencing pathway are targeted by these miRNAs, suggesting synchronization between silencing and pathogen defense pathways. The data illustrate a complex tasiRNA-mediated regulatory circuit that potentially modulates plant-microbe interactions. High levels of small RNAs were matched to over 60% of all ˜540 encoded Medicago NB-LRRs; in potato, a model for mycorrhizal interactions, phased siRNAs were also produced from NB-LRRs. The identification of a small number of miRNA triggers that can regulate an extremely large gene family by targeting highly conserved protein motif-encoding sequences is a new paradigm for miRNA function, because in plants miRNAs typically target just one or a few genes and those miRNA targets typically co-evolve with the miRNA, in a way that is distinct from the rest of the gene family. As described in detail in Zhai et al., 2011, Genes & Development 25:2540-53 (“Zhai 2011”), the data reveal complex tasiRNA-based regulation of NB-LRRs that potentially evolved to facilitate symbiotic interactions, and demonstrate miRNAs as master regulators of a large gene family, a new paradigm for miRNA function. The contents of Zhai 2011, including Supplemental Materials, are hereby incorporated in their entireties,

The present invention provides a method for generating a phased small RNA (phasiRNA) in a plant cell. The method comprises introducing into the plant cell an effective amount of a microRNA (miRNA).

The plant cell may be a cell from or in a plant. The plant may be a legume or a non-legume, preferably a legume. The legume may be selected from the group consisting of M. truncatula, soybeans, peanuts, and common beans. The non-legume may be selected from the group consisting of corn, rice, wheat, barley, oats, rye, sorghum, sugar cane, grapevine, almonds, apple, peach, sugar beet, tomato, potato, tobacco, cotton, lettuce, sunflower, melons, strawberries, and canola.

The term “microRNA” or “miRNA” used herein refers to a short ribonucleic acid (RNA) molecule from eukaryotic cells that regulates mRNA targets. A miRNA may function in a post-transcriptional manner by down-regulating target gene products involved in a variety of cellular processes. It may have 20 to 24 nucleotides (nt) in length, preferably 21 or 22 nt, more preferably 22 nt.

The miRNA may be any miRNA, naturally occurring miRNA or a derivative thereof. The miRNA may be isolated from any eukaryotic cell, preferably a plant cell, more preferably a legume cell. The term “derived from” used herein refers to an origin or source, and may include naturally occurring, recombinant, unpurified or purified molecules. An miRNA derived from an original miRNA may be a fragment or variant of the original miRNA. The term “variant” of an miRNA used herein refers to a short RNA having a nucleic acid sequence that is the same as the nucleic acid sequence of the miRNA except having at least one nucleic acid modified, for example, deleted, inserted, or replaced. A variant of an miRNA may have a nucleic acid sequence at least about 80%, 90%, 95%, or 99%, preferably at least about 90%, more preferably at least about 95%, identical to the nucleic acid of the miRNA.

The miRNA may have a sequence selected from SEQ ID NO: 1-166 or a derivative thereof. (Table 1). Other examples of the miRNA include of miR156, miR161, miR162, miR167, miR168, miR169, miR172, miR173, miR389*, miR390, miR393, miR472, miR482, miR772, miR828, miR1507, nniR1509, nniR1510, miR1512, miR1515, miR2089, miR2109, miR2118a, miR2118b, miR2118c, miR2597, miR5300, miR5754 and derivatives thereof. Preferably, the miRNA is a member of the miR1507 family, the miR2109 family, or the miR2118 family. Any sequence with four or fewer mismatches to the mature miRNA falls into the same family. Examples of the miR1507 family include gma-miR1507a, mtr-miR1507, gma-miR1507b, vun-miR1507a, vun-miR1507b, gso-miR1507a, gso-miR1507b, and gma-miR1507c. The mrR1507 family include miR472, miR482, miR2089, miR2118a, miR2118b, and miR2118c. Examples of the miR1507 family include gma-miR2109, and gso-miR2109. Examples of the miR2118 family include pvu-miR2118, mtr-miR2118, osa-miR2118a, osa-miR2118b, osa-miR2118c, osa-miR2118d, osa-miR2118e, osa-miR2118f, osa-miR2118g, osa-miR2118h, osa-miR2118i, osa-miR2118j, osa-miR2118k, osa-miR2118l, osa-miR2118m, osa-miR2118n, osa-miR2118o, osa-miR2118p, osa-miR2118q, osa-miR2118r, zma-miR2118a, zma-miR2118b, zma-miR2118c, zma-miR2118d, zma-miR2118e, zma-miR2118f, zma-miR2118g, gma-miR2118a, gma-miR2118b, and vun-miR2118. Preferably, the miRNA is miR482 or miR5300.

The term “phased small RNA” or “phasiRNA” used herein refers to a double-stranded ribonucleic acid (RNA) molecule from eukaryotic cells that interferes with the expression of a specific gene with a complementary nucleotide sequence. The phasiRNA may act in trans as tasiRNA or in cis as casiRNA, where trans indicates that the target of the phasiRNA is produced from the mRNA of a different gene than the phasiRNA, and cis indicates that the target of the phasiRNA is the mRNA of the same gene that produces the phasiRNA. The phasiRNA may have 20 to 25 nucleotides (nt) in length, preferably 21 nt. The miRNA is introduced into the plant cell in an amount effective to generate the phasiRNA in the plant cell. The miRNA may be introduced into the plant cell using conventional techniques known in the art. The introduction may be transient or permanent, preferably permanently. The miRNA may be introduced into the plant cell over a period of hours, days, weeks or months. It may also be introduced once, twice, or more times.

The transcript of a gene encoding a protein having a nucleotide binding (NB) and leucine rich repeat (LRR) domains may be cleaved. The NB-LRR encoding gene transcript may be cleaved once or twice, preferably once. The cleavage may be triggered by the miRNA, or one of the phasiRNAs triggered by a miRNA. The NB-LRR encoding gene may be a disease resistance gene. The NB-LRR encoding gene may be involved in modulating the interaction between a plant and a microbe. For example, the interaction may be a symbiotic interaction.

The effective amount of the miRNA may vary depending on various factors, for example, the sequence of the miRNA, the physical characteristics of the plant cell, the sequence of the desired phasiRNA, and the means of introducing the miRNA into the plant cell. A specific amount of the miRNA to be introduced may be determined by one using conventional techniques known in the art.

The phasiRNA may be generated from a locus called a PHAS gene in the plant cell. The PHAS gene may encode a NB-LRR protein. Examples of PHAS genes include DICER LIKE2 (DCL2), SUPPRESSOR OF GENE SILENCING3 (SGS3), and the eight Arabidopsis “TAS” (Trans-Acting siRNA) genes that generate miRNA-triggered secondary siRNAs (Howell et al. 2007).

The miRNA may be introduced into a cell in a plant. The plant may be a legume or a non-legume. The plant defense responses typically activated upon recognition of a microbe may be suppressed. Some plant genes may be suppressed. Recognition between the plant and the microbe may be enabled. Resistance of the plant to a microbe may be reduced. The microbe may be any microbe to which a NB-LRR may confer the plant resistance. For example, the microbe is a rhizobial strain. The plant may exhibit reduced resistance to a virus. The virus may be any virus to which a NB-LRR may confer the plant resistance. For example, the virus may be the tobacco N virus or cucumber mosaic virus. The plant may enter a symbiotic interaction with a microbe. The microbe may be a nitrogen-fixing strain such as a rhizobial strain, for example, Bradyrhizobium, or Frankia species. Nodulation or mycorrhizal interactions in the plant may be improved. The plant may exhibit improved seed germination, emergence, stand density, plant vigor, flowering, fruiting, biomass, plant growth and crop yield.

According to another aspect of the present invention, a composition comprising an effective amount of a miRNA for generating a phasiRNA in a plant cell is provided. The composition may further comprise a suitable carrier, diluent or excipient. Suitable carriers, diluent and other excipients are well known in the art.

The present invention may be used to modulate defense responses to microbes, particularly in the case of symbiotic interactions with beneficial microbes. To permit interactions with beneficial microbes, plants might need to suppress their defenses which would normally prevent the microbes from interacting directly with plant cells. This could be done via the small number of miRNAs newly identified which can trigger phased small RNAs, and these phased small RNAs create a highly interwoven silencing network that suppresses defense gene expression. Ultimately, this could potentially be used to transfer nitrogen-fixing properties to non-legumes like maize, rice, etc.—one of the “holy grails” of plant biology, since many billions of dollars are spent each year to manufacture nitrogen for application to non-legumes.

It may be possible to use this set of miRNAs in non-legume plants, together with receptors and pathway genes for the development of nodules, to promote beneficial, symbiotic interactions via suppression of defense genes. In the absence of the miRNAs and phased small RNAs newly identified, it may be impossible to successfully transfer the trait of nodulation to non-legumes, even though the genes that are required for the production of nodules could be transferred without the circuit we have identified; in this latter case, it is predicted that plant defenses would prevent the beneficial interactions.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1

### MicroRNAs as Master Regulators of the Plant NB-LRR Defense Gene Family Via the Production of Phased, Trans-Acting siRNAs Materials and Methods

### Plant Materials

M. truncatula A17 (Jemalong)

For developing seeds, flowers from greenhouse-grown plants were date-tagged at full bloom, and pods were selected for seed collection at 20 d after anthesis. For seedlings, seeds were surface-sterilized and sown on water agar in the dark as in Catalano et al. (2004), with whole seedlings collected 24 h post-sowing. Foliage, roots, and flowers were collected from plants grown aeroponically with complete nutrient supplementation within a controlled environmental chamber at 55% relative humidity and a 14-h, 22° C. day/10 h, 18° C. night cycle. Foliage and roots were collected 3 wk post-sowing, and flowers were collected at −1, 0, +1 tripping. For nodules, plants were grown aeroponically for 1 wk with ½×nutrient solution, transferred to nitrogen-free medium for 7 d, and inoculated with Sinorhizobium meliloti strain 2011 (Meade et al. 1982) 10⁶-CFU (colony-forming unit) plant-1 to induce nodule formation in a method modified from Catalano et al. (2004). Nodules of mixed developmental ages were collected at 14 d post-inoculation. Root knots were collected from an established root culture maintained on ½ MS salts, 20 g/L sucrose, 0.5 mg/L nicotinic acid, 0.5 mg/L pyridoxine.HCl, 0.4 mg/L thiamine.HCl, and 8 g/L Phytagar (GIBCO) after incubation in the dark for 6 wk post-inoculation at 28° C. with sterile Meloidogyne incognita eggs. Medicago/Glomus intraradices colonized and mock-inoculated roots were grown according to Liu et al. (2007).

G. max Williams 82 samples were collected as in Joshi et al. (2010).

P. vulgaris “Bat 93” seeds and flowers were collected from greenhouse-grown plants as for Medicago. Foliage and nodules were collected from aeroponically grown plants by the same method as Medicago, except that nodule formation was induced by the addition of Rhizobium leguminosarum by. viciae 3841, and nodules were collected and pooled at 7, 14, and 21 d post-inoculation.

A. hypogaea were grown in greenhouse conditions and induced to form nodules as in VandenBosch et al. (1994). Nodules and foliage were collected at 7, 14, 21 d post-inoculation with B. japonicum NC92.

Sequencing of Small RNAs from Legumes

Total RNA was isolated using Trizol reagents or plant RNA reagent, both from Invitrogen. M. truncatula small RNA libraries (except MTR01) were constructed and sequenced at Illumina; other libraries were constructed as previously described (C Lu et al. 2007) and sequenced on an Illumina GAIIx instrument at the Delaware Biotechnology Institute.

Twenty-one small RNA libraries were made from the materials described above, representing four legume species, including eight libraries from Medicago, seven from G. max (soybean), two from A. hypogaea (peanut), and four from P. vulgaris (common bean). Approximately 62 million small RNA sequences were obtained after removing adapters and low-quality reads, with trimmed lengths between 18 and 34 nt. After excluding small RNAs matching structural RNAs (t/rRNA loci), 12.1 million and 12.6 million reads were mapped to the Medicago genome (Mt3.5) (http://www.medicago.org) and the G. max genome (Gmax101) (Schmutz et al. 2010), respectively. Consistent with previous reports of plant small RNAs, small RNAs in all legume tissues are predominantly found in two sizes: 21 nt and 24 nt (Zhai 2011 Supplemental Fig. S8; Zhai 2011 Supplemental Table S11). We developed two Web sites and databases, using the Medicago and soybean genomes, to store and analyze all 21 libraries; we used these visualization tools extensively for this analysis. These Web sites are available at http://mpss.udel.edu/mt_sbs and http://mpss.udel.edu/soy_private.

### Small RNA Informatics Analysis

The miRNA prediction pipeline is outlined in Zhai 2011 Supplemental Figure S1, with details of the filters explained in the Zhai 2011 Supplemental Material.

We used CleaveLand to predict miRNA targets (Addo-Quaye et al. 2009). An astringent filter retained all matches with scores≦5; scoring was assigned by CleaveLand, and described previously (Allen et al. 2005). The PARE data were integrated using the pipeline described in the Zhai 2011 Supplemental Material.

Phasing analysis was performed as described previously (De Paoli et al. 2009). As a final check of loci with phasing scores≧15, scores and abundances of small RNAs from each high-scoring locus were graphed and checked visually to remove false positives such as miRNAs with numerous low-abundance peaks that could incorrectly pass our filters. We also manually removed unannotated tRNA and rRNA-like loci with high phasing scores because of their high small RNA levels.

Comparative Analysis of miRNAs from Diverse Plant Species

We used libraries from 30 diverse species plus the legume libraries. The sequences of the eight miRNAs of interest were searched for exact and near matches, allowing three mismatches and up to a 2-nt shift at either the 5′ or 3′ end or both. Small RNAs 20-24 nt in size and represented by at least two reads in a library were analyzed. Alignments were performed using SeqMap (Pawlowski et al. 2004) followed by output filtering and reformatting by custom-written PERL scripts. Heat maps were created using customized PERL scripts and the Inkscape vector graphics software (http://www.inkscape.org).

Synteny Analysis of the M. truncatula and Soybean PHAS Loci

To identify orthologous pairs of PHAS loci in the Medicago (Mt3.5) and soybean (Gmax101) genomes, we performed pairwise alignments by BLASTN with default parameters, Pairs of sequences with high similarity (identity>60% and aligned coverage>500 bp) were selected to check for synteny between Medicago and soybeans by comparing their locations to syntenic blocks between Medicago and soybeans detected by SyMAP (Soderlund et al. 2011). Because of the possibility that some small regions of colinearity may have been masked by a larger region, MUMMer (Kurtz et al. 2004) was used to align each pair of 1-Mb extended regions (500 kb on each side of each PHAS locus) in order to further identify sequences that are in the region of colinearity.

Phylogenetic Analysis of the Family of NB-LRR Proteins Encoded in the M. truncatula Genome

We extracted predicted protein sequences for 312 TNLs and 385 CNLs from the Medicago genome sequence, including unassembled contigs. The unassembled contigs were not integrated into the genome, and thus were not used for phased siRNA or any small RNA analysis. A total of 542 NB-LRR proteins were encoded in the assembled Medicago genome and used for small RNA analysis; the subset of these NB-LRRs with a high level of small RNAs (≧50 TPM) is denoted in the cladograms with red dots. The remaining 155 NB-LRRs presumably map to gaps in the assembled genome sequence; these are denoted in the cladograms by gray dots.

The cladograms were constructed using maximum parsimony based on only the nucleotide-binding site of the NB-LRR proteins (the conserved NB-ARC domain). Separate TNL class and CNL class trees were rooted with the nearest neighbor in the other class determined from a joint tree.

### GenBank Accession Numbers

The GenBank Gene Expression Omnibus (GEO) accession numbers for these data are GSE28755 for the small RNA data from 30 diverse plants (also found at http://smallrna.udel.edu) and GSE31061 for the legume small RNAs and PARE sequencing results. The legume data are also available at http://mpss.udel.edu/mt_sbs and http://mpss.udel.edu/soy_sbs.

### Results

miRNA Identification in Medicago truncatula and Soybeans

Although numerous miRNAs have been identified from legume species, the availability of complete genome sequences provides an opportunity for identification of poorly conserved or other novel miRNAs. In a recent release of miRBase (version 16), a total of 383 miRNA genes have been annotated in M. truncatula, 203 have been annotated in Glycine max (soybean), and many fewer have been annotated in Arachis hypogaea (peanut) and Phaseolus vulgaris (common bean). We believed that miRNA identification is not yet saturated in these species. Therefore, we used a larger set of libraries and tissues, combined with the more complete genome sequences of M. truncatula and soybeans, comparative genomics methods, a powerful new miRNA prediction pipeline, and large-scale validation of target cleavage to identify new legume miRNAs, phased or trans-acting-like small RNAs, and hc-siRNAs. Twenty-one small RNA libraries were made from tissues of four legumes, including M. truncatula, soybeans, peanuts, and common beans (Zhai 2011 Supplemental Table S1). These libraries included ˜62 million small RNA reads. Given the absence of peanut and common bean genomes, we used those species' data for comparative analysis and focused on the M. truncatula and soybean data.

Using the M. truncatula libraries, we applied an informatics pipeline for filtering plant miRNAs from the complete set of small RNAs. Our input to this pipeline was 9,282,720 distinct small RNAs sequenced from eight M. truncatula libraries (Zhai 2011 Supplemental Fig. S1); among these sequences were 98 different, known, mature M. truncatula miRNA sequences of the 383 annotated in miRBase version 16. The other 285 annotated miRNAs not sequenced may be weakly expressed in the tissues we sampled or are not real miRNAs. After passing the data through the pipeline (see Zhai 2011 Supplemental Material), 90 M. truncatula miRNA candidates remained, generated from 137 precursors, which were compared against miRBase version 16 to identify high-similarity homologs. Excluding 26 M. truncatula miRNAs that were previously annotated, 22 sequences were found with >85% similarity to known plant miRNAs, leaving 42 new miRNA candidates from 51 precursors (Zhai 2011 Supplemental Table S2). A similar strategy for the soybean genome and small RNA libraries started from 6,133,687 distinct small RNAs and 166 annotated soybean miRNAs and identified 40 new miRNA candidates from 45 precursors (Zhai 2011 Supplemental Table S3). New members of known miRNA families with loci not annotated in version 3.5 of the M. truncatula genome and new miRNA candidates identified from the computational filters applied to the eight M. truncatula libraries are set forth in Zhai 2011 Supplemental Table S2B and S2C. Similarly, new members of known miRNA families with loci not annotated in M. max genome and new miRNA candidates identified from the computational filters applied to the seven G. max libraries are set forth in Zhai 2011 Supplemental Table S3B and S3C. These sequences are shown in Table 1.

We noticed that many M. truncatula and soybean miRNA sequences were 22 nt in length, some of which were quite abundant. Recent reports show that 22-nt miRNAs are necessary and sufficient to trigger phasing at tasiRNA loci (Chen et al. 2010; Cuperus et al. 2010). In our M. truncatula data, the 22-nt miR1507, miR1509, miR2109, miR2118, and miR2597 were highly abundant (Zhai 2011 Supplemental Table S2). Several of these mature miRNAs previously were annotated with sizes other than 22 nt, and, based on our libraries, we believe those earlier size annotations are incorrect. For example, in miRBase, miR2109 is annotated as 20 nt, miR2597 is annotated as 21 nt, and miR1509 is annotated as 21 nt. In M. truncatula, we predicted eight new 22-nt miRNAs (Zhai 2011 Supplemental Table S2C), plus we found 22-nt variants of previously described 21-nt miRNAs (miR156 and miR169), and we found 22-mers that passed our miRNA filters, corresponding to the miRNA* positions of conserved miRNAs (two copies of miR169*, plus miR398*) (Zhai 2011 Supplemental Table S2B). This abundance of 22-nt miRNAs is in contrast to Arabidopsis, which has few 22-nt mature miRNAs annotated in miRBase, most of which occur at low abundances: miR173 (targeting TAS1 and TAS2), miR393, miR472, and miR828 (targeting TAS4). The soybean genome included at least 28 loci producing 22-nt mature miRNAs (Zhai 2011 Supplemental Table S3). miR1507, miR1509, and miR2118 are highly abundant 22-nt miRNAs in both M. truncatula and soybeans (Zhai 2011 Supplemental Tables S4,S5). Three differences of abundant 22-nt miRNAs were observed in the M. truncatula-soybean comparison: miR2597 was abundant in M. truncatula and not found in soybeans, miR1512 was abundant in soybeans but not found in M. truncatula, and miR2109 was abundant in both species but was 22 nt in M. truncatula and predominantly 21 nt in soybeans. The conservation and expression of these 22-nt miRNAs in peanuts and common beans are described below. We concluded that 22-nt miRNAs are both numerous and expressed abundantly in legumes.

Identification and Validation of M. truncatula miRNA Targets

We predicted potential miRNA targets and integrated the matches with empirical cleaved mRNA data to identify valid miRNA targets. The 90 unique M. truncatula miRNA candidate sequences from 137 precursors that passed our filters were searched against both genome and cDNA sequences. Predicted matches with penalty scores≦5 (>50,000 predicted miRNA-target pairs in the genome, and >15,000 pairs from cDNAs) were combined with a PARE (parallel analysis of RNA ends) library (German et al. 2008) made from M. truncatula flower tissue (Zhai 2011 Supplemental Table 51). The observation of several conserved miRNA-target pairs (miR156-SPL, miR172-AP2, miR167-ARF8, and miR390-TAS3) suggested that library quality was sufficient for validation of miRNA targets; these targets exhibited precise, high-abundance cleavage products at the predicted target sites (Zhai 2011 Supplemental Fig. S2).

To detect novel mRNA targets in M. truncatula, we used several stringent filters for the PARE data (see the Materials and Methods). We confirmed 144 cleavage sites from 89 genes and 30 intergenic regions, targeted by 46 different miRNAs (Zhai 2011 Supplemental Table S4A). These targets include a broad set of genes not previously known to be targets of miRNAs. While most target genes were cleaved by only one miRNA at a single recognition site, we identified two target sites for miR1509 in Medtr7g012810; this is significant because in the Arabidopsis TAS3 gene, two “hits” by the 21-nt miR390 are required to trigger the production of tasiRNAs (Axtell et al. 2006). The combination of the stringent filters to identify the miRNA candidates and the conservative methods of experimental validation with the PARE data allowed us to confirm 49 functionally validated M. truncatula miRNAs in flower tissue, and thus these were added to miRBase. We also propose to correct several M. truncatula miRNAs that we believe were annotated previously with incorrect sizes or mature sequences.

Identification of tasiRNA-Like phasiRNA Loci in M. truncatula and Soybeans

The identification of two target sites for miR1509 in Medtr7g012810 led us to ask how many M. truncatula miRNAs might trigger tasiRNAs. The eight Arabidopsis “TAS” genes generate miRNA-triggered secondary siRNAs in a 21-nt “phased” pattern (Howell et al. 2007). We previously refined and applied a computational approach to evaluate the phasing pattern of small RNAs (De Paoli et al. 2009). The small RNAs identified from this algorithm are phased but do not necessarily function in trans (or even in cis); therefore, we call these phased siRNAs “phasiRNAs.” Since tasiRNA-generating loci are TAS genes, we propose that phasiRNA-generating loci are PHAS genes.

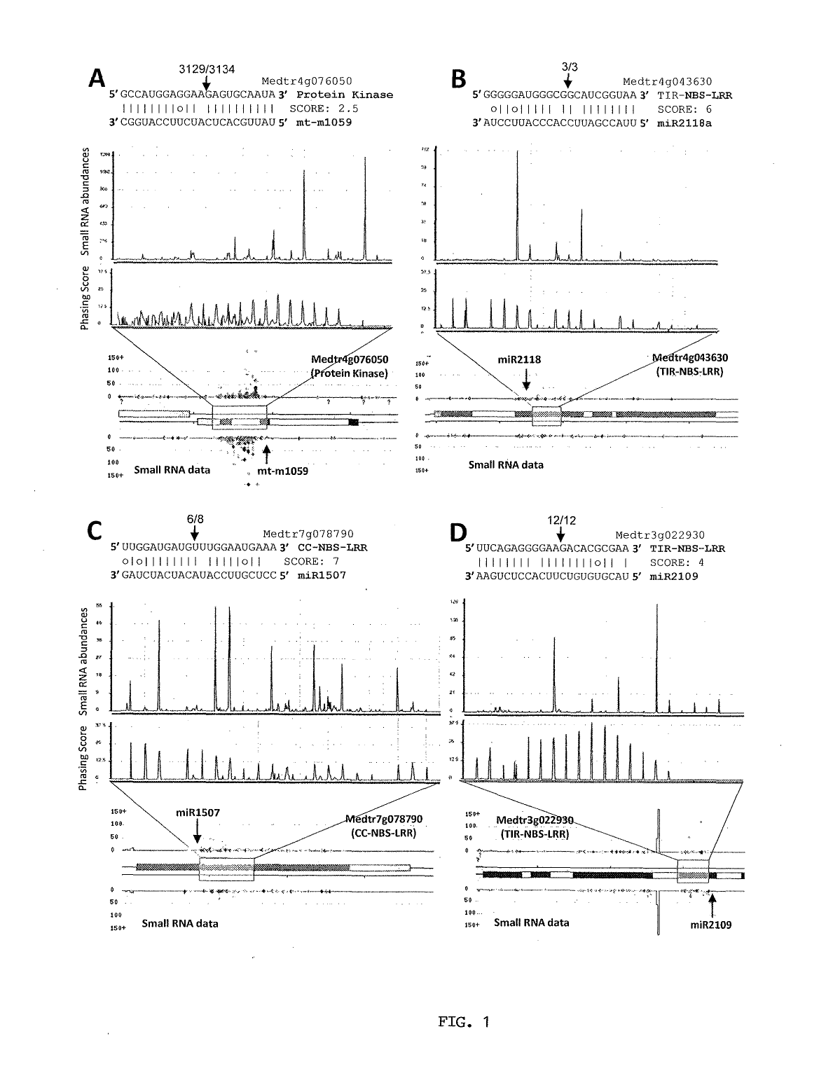

We applied this same algorithm to the M. truncatula and soybean genomes for our libraries. Using Arabidopsis as a control, all loci with a phasing score≧15 in any one library are considered above background (summarized in Zhai 2011 Supplemental Table S5). PhasiRNAs with only 21-nt intervals were identified from a large number of loci (after removing false positives) (see the Materials and Methods). This set included 112 genes and two intergenic regions in M. truncatula (examples in FIG. 1; Zhai 2011 Supplemental Table S6) and 26 genes and 15 intergenic regions in soybeans (Zhai 2011 Supplemental Table S7). MtTAS3 is the only M. truncatula phased locus that has been described previously (Jagadeeswaran et al. 2009), and the remainder are novel PHAS loci that have not been previously described in other species.

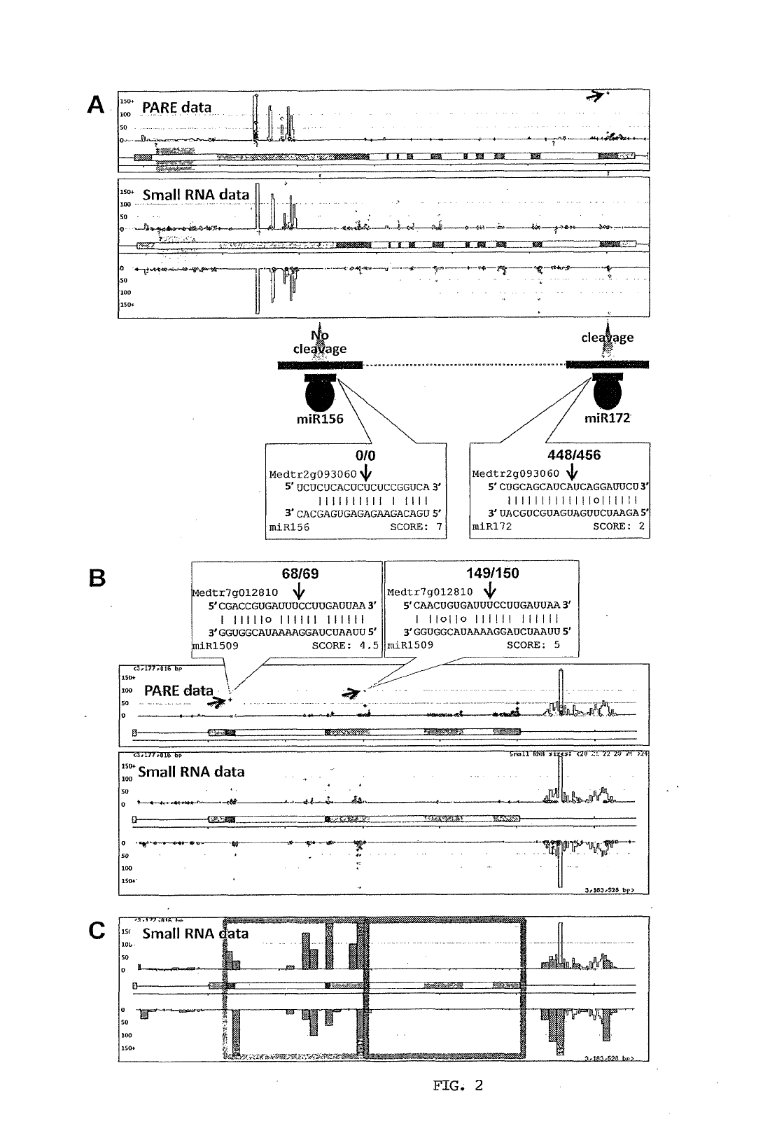

We integrated our miRNA lists, target prediction, and the PARE data to identify the triggers for the M. truncatula PHAS loci. We were able to identify the miRNA triggers for most PHAS loci (summarized in Table 2; Zhai 2011 Supplemental Table S6). At least 77 of the 114 M. truncatula PHAS loci (˜68%) are triggered via single cleavage of a 22-nt miRNA trigger; we call this “1₂₂” for a single-target, 22-nt miRNA trigger event. The majority of these PHAS loci were triggered by a few high-abundance 22-nt miRNAs (miR1507, miR1509, miR2109, and miR2118a/b/c). There were just a few exceptions to the predominance of 1₂₂ PHAS loci. We also identified a novel two-hit (2₂₁) PHAS locus (the second known example, in addition to TAS3), an AP2-like gene (Medtr2g093060). Consistent with the two-hit model, Medtr2g093060 includes a conserved, cleaved miR172 target site (Aukerman and Sakai 2003), plus a predicted noncleaving and highly degenerate miR156 target site (FIG. 2A). While the miR156 target site has extensive mismatches and a poor score, no other target sites were identified in the upstream region for any other miRNA. The miR172 upstream direction of the phasiRNAs and the validation of the cleaved site in the PARE data resemble TAS3, consistent with the two-hit model of phasiRNA biogenesis from this transcript. Two other related miR172 targets identified in the M. truncatula genome (Medtr7g100590 and Medtr4g061200) do not have this miR156 site and thus showed no evidence of phasiRNAs. Soybean orthologs of Medtr2g093060 (Glyma13g40470 and Glyma15g04930, two due to genome duplications) both have conserved miR172 target sequences but lack any conservation of the untranslated region (UTR) that includes the miR156 site and showed no evidence of phasiRNA production; the miR156 target sequence was also not conserved in Lotus japonicas. This unique example of an AP2-like M. truncatula PHAS gene suggests that the spontaneous acquisition of a new miR156 or other yet-to-be-identified 5′ noncleaving target site is a recent evolutionary event. Another exception to the 1₂₂ PHAS loci that predominate was a 2₂₂ PHAS gene demonstrating double cleavage by a 22-nt miRNA (FIG. 2B); Medtr7g012810 is targeted by miR1509 at two cleaved sites (Zhai 2011 Supplemental Tables S6, S8). Nearly all small RNAs were found between the two cleaved target sites, with a near absence of small RNAs 3′ of the poly-A-proximal target site (FIG. 2C). Small RNAs generated immediately downstream from the 5′ cleavage site, as well as those immediately upstream of the 3′ cleavage site, were perfectly in phase with their respective cleavage sites, consistent with bidirectional processing of the central portion of this 2₂₂ PHAS locus. In summary, the M. truncatula small RNA and PARE data identified many 1₂₂ loci, two 2₂₁ loci, and one 2₂₂ PHAS locus.

Two genes known to be involved in small RNA biogenesis (DICER-LIKE2 [DCL2] and SUPPRESSOR OF GENE SILENCING3 [SGS3]) were also identified as PHAS genes (Zhai 2011 Supplemental Fig. S3). The DCL2 trigger is predicted to be miR1507 in M. truncatula but miR1515 in soybeans (validated by Li et al. 2010), with the phasiRNAs initiating from different sites in the orthologs, consistent with different miRNA target sites (Zhai 2011 Supplemental Fig. S3A). PhasiRNAs were identified from the soybean ortholog of SGS3 (Glyma05g33260 [GmSGS3]), but not the three paralogs of SGS3 in M. truncatula; GmSGS3 was previously validated as a target of a miR2118 family member (Song et al. 2011). The recruitment of genes involved in phasiRNA biogenesis as sources of phasiRNAs suggests a feedback mechanism reminiscent of the regulation of Arabidopsis AGO1 and DCL1 by miR168 and miR162, respectively (Xie et al. 2003; Vaucheret et al. 2004, 2006).

We identified a novel, potentially noncoding, 1₂₂ PHAS locus in M. truncatula of particular interest because the phasiRNAs were the most abundant in our data set (10-fold higher than TAS3, the most abundant conserved TAS locus), the PARE sequences matched on both strands, and the phasing score was extremely high (Zhai 2011 Supplemental Table S6; Zhai 2011 Supplemental Fig. S4). This intergenic region on Chr. 2 has very weak similarity to the PPR family of genes, suggestive of the Arabidopsis TAS2 locus that targets PPR genes (Montgomery et al. 2008b). TAS2 is triggered by miR173, but the trigger of this M. truncatula PHAS gene is a novel 22-nt miRNA candidate at moderate abundances in all of our M. truncatula libraries but with no known genomic origin (perhaps due to gaps in the genome). The noncoding precursor and tasiRNAs that initiate cleavage on numerous targets (Zhai 2011 Supplemental Table S7) are reminiscent of TAS genes; we named this locus PHAS_IGR1. The abundant PARE reads from both strands (Zhai 2011 Supplemental Fig. S4) are not predicted for RDR6 products, since PARE reads are derived from poly-A mRNA. Another unusual aspect of this locus is the evidence of cleavage by siRNAs acting in cis, also previously reported at TAS3 in which the −D2 siRNA cleaves the primary TAS3 transcript out of phase and without producing secondary siRNAs (Allen et al. 2005; Jagadeeswaran et al. 2009). The cis-targeting siRNAs might serve as a negative feedback loop in overall phasiRNA production.

To systematically determine whether the M. truncatula phasiRNAs function in cleavage, we again examined the PARE data. By definition, tasiRNAs should function to direct cleavage (or silencing) at second sites (e.g., in trans), although data suggest some may function in cis (Allen et al. 2005). Using the top five phasiRNAs by abundance from each of the 114 PHAS loci and integrating genomic target predictions and our PARE data, a stringent cutoff (see the Materials and Methods) identified ˜2000 sites whose cleavage is guided by these 570 phasiRNAs, including numerous cases of cis regulation (Zhai 2011 Supplemental Table S8). Therefore, we identified many verifiable trans- and cis-acting siRNAs produced from the large number of legume PHAS loci.

Twenty-Two-Nucleotide miRNAs as Master Regulators of Legume NB-LRR-Encodinq Genes and Generators of phasiRNAs

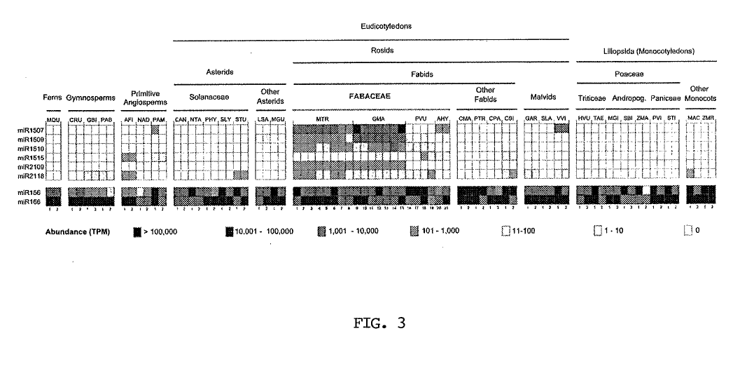

A feature of the legume phasiRNA loci was the preponderance of NB-LRR-encoding genes, including 79 of 112 M. truncatula phasiRNA loci. We call these genes phasi-NB-LRRs, or pNLs. We found that just three 22-nt miRNA families (miR1507, miR2109, and miR2118) are responsible for the initiation of the phasiRNAs at 74 of the 79 pNLs (Zhai 2011 FIG. 3A; Zhai 2011 Supplemental Table S6). miR1507 “specializes” in targeting CC-NB-LRR (CNL) genes, with strong complementarity to the encoded kinase-2 motif, centered near a highly conserved tryptophan (W) (Zhai 2011 FIG. 3A). miR2109 targets the TNL class, matching the encoded TIR-1 motif of the TIR domain (described in Meyers et al. 1999). The three-member miR2118 family (miR2118a/b/c; miR2118c is renamed from miR2089) targets sequences encoding the most well-conserved NB-LRR motif, the P-loop (Zhai 2011 FIG. 3A; Meyers et al. 1999). miR2118a and miR2118c preferentially target TNL genes, while miR2118b almost exclusively targets CNL genes (Zhai 2011 Supplemental Table S6). Thus, a specialized group of miRNAs targets conserved domains of NB-LRRs in legumes.

Next, we asked whether the pNLs represent a single, distinct clade within the broader phylogenetic group of NB-LRRs. We mapped the pNLs and their miRNA triggers onto cladograms of the TNL and CNL groups from M. truncatula (Zhai 2011 FIG. 3B; Zhai 2011 Supplemental Fig. S5). pNLs were found to be widely distributed across both the CNL and TNL groups. The triggers for the pNLs were similarly distributed, with no apparent pattern or grouping in the tree; this was especially evident in the TNL group (Zhai 2011 FIG. 3B). With many small RNAs (>50 TPM [transcxripts per million]) matched to >60% of the ˜540 NB-LRRs in the assembled Medicago genome (Zhai 2011 FIG. 3B; Zhai 2011 Supplemental Fig. S5A; Zhai 2011 Supplemental Table S9), there may be many more pNLs. An analysis of all NB-LRR-associated small RNAs demonstrated that they were predominantly 21-mers, and the majority of NB-LRRs have more 21-nt small RNAs than any other size class (Zhai 2011 Supplemental Fig. S5B,C). This indicates that the silencing of NB-LRRs by phased small RNAs is a phenomenon that occurs broadly across the gene family via a small number of miRNA triggers, each of which is effective against distantly related members of the family. These unique attributes are the result of the targeting of sequences encoding a conserved protein motif by a miRNA, an activity that has not been previously described but may provide flexibility yet rigidity to target a large number of diverse yet related genes.

We next searched for evidence of soybean pNLs, identifying 13 pNL loci. Only 13 of 41 soybean PHAS loci are pNLs (Zhai 2011 Supplemental Table S7). Glyma16g33780 produces limited phased 21-nt siRNAs; this is the Rj2/Rfg1 allelic pair, a TNL of known function that defines rhizobial host specificity (Yang et al. 2010). PhasiRNAs from this gene and many pNLs match more than one gene; those from Rj2/Rfg1 averaged more than four matching genomic loci. This interconnectivity between pNLs would facilitate an extensive and coordinated regulatory network. In the absence of soybean PARE data, we assessed whether the soybean pNL triggers were related to the M. truncatula pNL triggers. The highly expressed M. truncatula miR1507 is well conserved in soybeans and is predicted to initiate pNLs. The M. truncatula 22-nt miR2109 is predominantly 21 nt in soybeans, suggesting that it may not initiate soybean pNLs (vs. triggering≧14 pNLs in M. truncatula), consistent with fewer pNLs in soybeans. In soybeans, the miR2118 family is slightly more complicated, because this miRNA is multicopy and is the star sequence to miR482. We checked to see whether the M. truncatula and soybean pNLs are found in syntenic locations indicative of an origin early in legume evolution; only two pairs were found in syntenic blocks (Mt-TAS3:Gma-TAS3a and Mt-TAS3:Gma-TAS3b) and no pNLs were syntenic (Zhai 2011 Supplemental Table S10). This finding and the broad phylogenetic distribution of miRNA targets and pNLs suggest a dynamic nature to the subset of NB-LRRs that are pNLs.

Evolutionary Conservation of 22-Nt miRNA Families from Legumes

We examined sequence conservation among 30 diverse and agronomically relevant plant species, including lower plants and basal angiosperms, for homologs of the six most-abundant and PHAS targeting 22-nt legume miRNA families: miR1507, miR1509, miR1510, miR1515, miR2109, and miR2118. The common bean and peanut data demonstrated that most of the 22-nt miRNAs were highly abundant within legumes (FIG. 3; Zhai 2011 Supplemental Fig. S6). Comparisons across the larger set of species showed these miRNAs present at low abundances (<10 TPM) (FIG. 3) in many species; those levels are low enough that the precursors and expression will need to be verified when the genomes become available. Analysis of the grape, maize, and potato genomes showed that some but not all of these small RNAs come from predicted miRNA-like hairpins. At a more robust threshold of abundance of 11-100 TPM, we found that miR1507, miR1509, miR1515, and miR2118 are present in nonleguminous species (FIG. 3). miR1507 is highly abundant in grapes and avocados, and miR2118 was broadly represented and relatively abundant (≧100 TPM) outside of legumes, including moderate signals in all four libraries of the Ginko (GBI in FIG. 3) and Norway spruce (PAB in FIG. 3). This presence in two gymnosperms that date back>250 million years suggests that these phased siRNAs may represent an ancient regulatory mechanism.

miR2118 was also abundant in potatoes (FIG. 3), and the potato genome was recently sequenced (Xu et al. 2011), so we examined whether this genome also contains many phasiRNAs or even pNLs. At the same cutoff used previously (≧15), the three potato small RNA libraries identified 36, 33, and 43 PHAS loci (Zhai 2011 Supplemental Table S5). Examination of a subset of these phased loci identified numerous pNLs (Zhai 2011 Supplemental Fig. S7A). Many of these phasiRNAs also matched to N8-LRRs clustered on Chr. 11 (Zhai 2011 Supplemental Fig. S7B), a region of the chromosome known to contain many active disease resistance genes (Gebhardt and Valkonen 2001). Our analysis of the potato small RNA data suggested that the triggers of these pNLs also include the recently described 22-mer miR5300 (Mohorianu et al. 2011), which targets the encoded P-loop of NB-LRRs within a nucleotide of the same site as miR2118 (Zhai 2011 Supplemental Fig. S7B). Together with a recent report of pNLs in grapes (http://www.intl-pag.org/19/abstracts/W84_PAGXIX_{—}525. html), these data suggest that 22-nt pNL-targeting miRNAs evolved early in plants and that pNLs are found in nonleguminous species.

### DISCUSSION

We identified a large number of novel miRNAs, and the analysis of these miRNAs and their targets has substantially expanded our understanding of small RNA biology in plants. The extensive network of phasiRNAs is apparently absent in Arabidopsis, yet we believe that the implications of our data go beyond legumes, as we also demonstrated the presence of many PHAS loci and their miRNA triggers in other plants.

Legume miRNAs have Evolved in Unique Ways

We characterized novel miRNAs in legumes and identified and validated novel legume targets. By integrating PARE data with small RNA data and novel bioinformatics analyses, we identified 42 new miRNA candidates from 51 precursors in M. truncatula and 40 new miRNA candidates from 45 precursors for soybeans. Our analysis demonstrated that both the M. truncatula and soybean genomes encode a larger set of 22-nt miRNAs than any plant genome described to date. This size class of miRNAs has an innate ability to trigger phased small RNA cascades in plants (Chen et al. 2010; Cuperus et al. 2010). The 22-nt miRNAs are produced from at least 21 loci in the M. truncatula genome and 28 loci in the soybean genome, whereas Arabidopsis generates just a few known 22-nt mature miRNAs (Cuperus et al. 2010), most of which are weakly expressed. Many of these legume 22-nt miRNAs are highly abundant in the tissues that we characterized. Many of the new 22-nt miRNAs we identified have no or few validated target sites in this set; it is possible that these function to trigger phasiRNAs in tissues we did not examine. The profusion in these legume genomes of 22-nt miRNAs and the phasiRNAs that they initiate suggests that the tasiRNA pathway is more broadly useful as a genetic regulatory circuit, raising the question of whether the relatively low number of phased loci in Arabidopsis is a general feature of plant genomes or is exceptional. This can be addressed by the analysis of phased small RNAs across the increasingly large number of sequenced plant genomes.

Legume phasiRNAs and the General ‘Rules’ for their Biogenesis in Plants

We identified a large number of loci that fit with the original two-hit (21-nt trigger, or “2₂₁”) or single-hit (22-nt trigger, or “1₂₂”) models for tasiRNA biogenesis (for review, see Allen and Howell 2010); the diversity of phasiRNAs described in our study has implications for understanding the general properties of their biogenesis. For example, we identified a new “two-hit” plant PHAS locus (FIG. 2A), demonstrating that this pathway is not unique to TAS3, a well-conserved but heretofore unique plant developmental regulatory circuit. In fact, evidence of phased siRNAs generated from multiple target sites in a transcript have been reported for the PPR-encoding targets of TAS2 siRNAs and miR161 (Howell et al. 2007; Chen et al. 2010). In our data, the paired configuration of two 21-nt miRNA target sites (cleaving [miR172] and noncleaving [miR156]) is similar to TAS3. Like TAS3, the mRNA fragment 5′ of the miRNA cleavage site in the AP2-like transcript is converted into phased small RNAs, confirming the distinct 5′ directionality of processing under the two-hit model (Axtell et al. 2006). Like miR390, miR172 falls into the minority of conserved plant miRNAs that lack a 5′ U; since the 5′-terminal nucleotide is important for sorting miRNAs and loading onto different Argonaute proteins (Mi et al. 2008), it is possible that this characteristic is functionally important for the two-hit triggers. Montgomery et al. (2008a) demonstrated that miR390 is selectively bound by AGO7, and this AGO7-miR390 complex is required at the noncleaving TAS3 target site for tasiRNA biogenesis; they inferred that AGO7 binding may be a requirement for the two-hit tasiRNA pathway. In contrast, miR156, the noncleaving trigger in the M. truncatula AP2-like RNA, is known to be bound by AGO1 in Arabidopsis, suggesting that AGO7 loading may not be necessary for two-hit biogenesis of tasiRNAs or that miR156 is bound by AGO7 in M. truncatula.

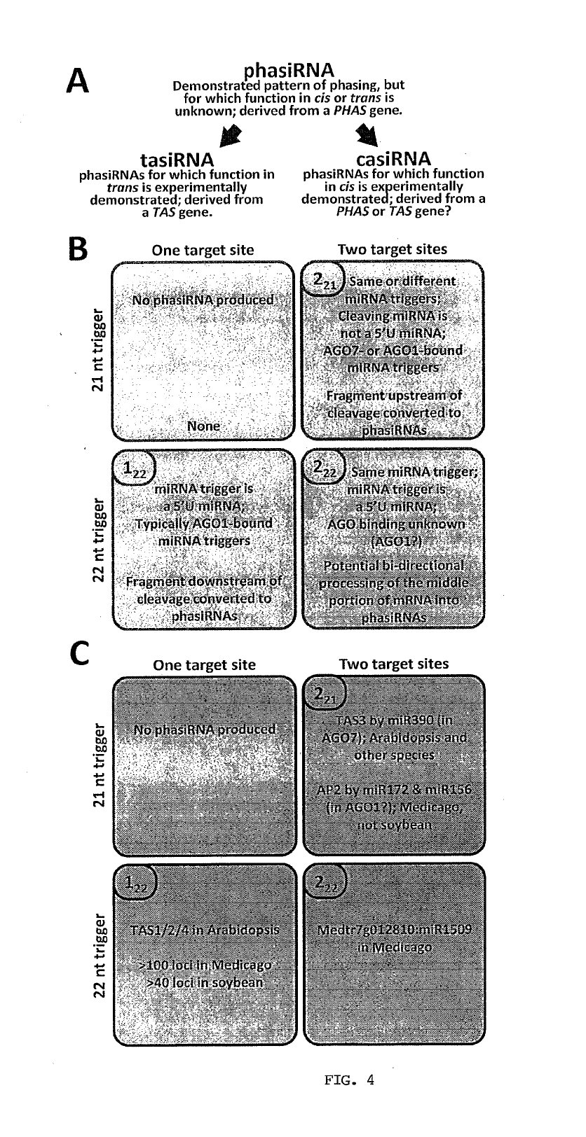

Our work has expanded our understanding of tasiRNA triggers. This diversity of phasiRNA loci suggests a need for a better organizational scheme to describe these secondary siRNAs. Here we propose to use the term “phasiRNA,” as we believe that a tasiRNA cannot be called such without evidence of targeting activity in trans. There is evidence of cis activity in TAS3, and this functional self-targeting small RNA is conserved in Arabidopsis and M. truncatula (Allen et al. 2005; Jagadeeswaran et al. 2009); phasiRNAs targeting their source locus like this could be “casiRNAs.” We summarized these classes of phasiRNAs and their precursors in FIG. 4A.

In both the two-hit model and a single-hit model for tasiRNA biogenesis, cleavage occurs at only one site; the two “hits” of the two-hit model include one uncleaved target site (Axtell et al. 2006; for review, see Allen and Howell 2010). In our study, a new class was represented by a 2₂₂ PHAS gene. PhasiRNAs at Medtr7g012810 are triggered after double cleavage by a 22-nt miRNA. Because 22-nt miRNAs trigger phasiRNA production in the poly-A-proximal fragment of PHAS transcripts, and because we observed high-abundance small RNAs between the two target sites (FIG. 2B,C) yet in phase with both the 5′ cleavage site (downstream phased) and the 3′ cleavage site (upstream phased), we infer a synergistic effect of having two adjacent 22-nt target sites. This suggests that a 2₂₂ cleavage product is processed inwardly from the two cleavage sites in both the cap- and poly-A-proximal directions, consistent with the directionality of processing for both the single- and two-hit models (Allen and Howell 2010). One alternative explanation for the phasiRNAs at this locus is that the two target sites function independently, whereby we might have expected similar levels of small RNA 3′ of both target sites; instead, almost no small RNAs were found 3′ of the 3′-most target site (brown box in FIG. 2C), suggesting that this 22-nt target site does not function as an independent 1₂₂ site. Based on the conservation of miR1509 across legumes, 2₂₂-based PHAS genes are likely moderately conserved.

Taken together, these results allow us to refine earlier models for phasiRNA biogenesis. Prior work demonstrated that phasiRNA production is dependent on either 2₂₁ or 1₂₂ targeting, resulting in either cap- or poly-A-proximal processing (FIG. 4B; Allen et al. 2005; Axtell et al. 2006; Montgomery et al. 2008b; Chen et al. 2010; Cuperus et al. 2010). We summarized the loci that exemplify these cases in FIG. 4C. Experiments have demonstrated that the noncleaving site both is conserved in flowering plants (Axtell et al. 2006) and cannot be replaced by a cleaving site in the 2₂₁ model (Montgomery et al. 2008a; Felippes and Weigel 2009), although double cleavage has been observed in mosses (Axtell et al. 2006). We showed that 2₂₂ loci are processed into phasiRNAs almost exclusively between two cleaved target sites (FIG. 2B,C). These data suggest two points: First, the two-hit 2₂₂ pathway is epistatic to the hypostatic single-hit 1₂₂ mode of processing; and second, double cleavage can strictly delimit the boundaries of phasiRNA production. Although we do not know the directionality of the processing, if the 22-nt miRNAs trigger 2₂₂ phasiRNAs, as in the 1₂₂ PHAS transcripts, processing may occur from both cleaved ends toward the center of the double-cleaved transcript.

### NB-LRRs are Targets of an Extensive Small RNA Regulatory Network

Our study demonstrated that NB-LRRs are targeted by multiple, independent miRNA families, and each of these miRNAs targets a region encoding highly conserved protein motifs. At least three miRNA families in legumes are predicted to target transcripts from hundreds of NB-LRR-encoding genes, and phased small RNAs are generated from at least 79 M. truncatula pNL genes. The 22-mer pNL trigger miR2109 in M. truncatula is mostly 21 nt in soybeans; size diversification by altering the proportion of 21-nt versus 22-nt variants of a miRNA may allow flexibility in the degree of silencing of target genes due to differences in their ability to trigger phasiRNAs that amplify post-transcriptional silencing. The larger family of plant NB-LRR targeting miRNAs includes the 22-nt miR472, which was first identified in poplar (S Lu et al. 2007) and later found in Arabidopsis (called miR772 at that time) (Lu et al. 2006), in which it was demonstrated to target and cleave NB-LRR transcripts at P-loop-encoding regions. Based on a simple sequence comparison, both miR472 and miR1510* are closely related to miR482 and miR2118. The 21-nt miR1510 annotated in soybeans and abundant in our M. truncatula libraries is also predicted to target NB-LRR-encoding transcripts (Valdes-Lopez et al. 2010). Most plant miRNAs target many fewer genes than the number of targets of the pNL triggers; this suggests that the pNL-triggering miRNAs are also quite unusual because they target conserved motifs, regulating an extensive gene family. In the 1990s, degenerate oligos were widely used to amplify large sets of NB-LRRs (Michelmore 1996); it now appears that nature beat scientists to the punch, taking advantage of the “degeneracy” of miRNA-target interactions to broadly interact with the NB-LRR gene family.

pNL triggering may evolve rapidly. The pNLs are distributed throughout the CNL and TNL families of M. truncatula and are well represented in soybeans. We found no evidence for synteny among pNLs in these two legume genomes. One interpretation of the lack of synteny between the M. truncatula and soybean pNLs is that miRNA target sites may be gained or lost with relative ease by substitutions in the few nucleotides for which changes would not disrupt the protein motif but would disrupt the miRNA-mRNA interaction. While deeper sequencing may yet identify paralogous legume pNLs, the pNL subset of NB-LRRs may evolve relatively quickly depending on microbial selection pressures.

Is this phenomenon of pNLs specific to the legumes? In Arabidopsis, only one NB-LRR, a TNL, was identified as generating phasiRNAs (Howell et al. 2007); our reanalysis of this locus with much deeper data did not confirm this, yet there are many 21-nt siRNAs at this locus reported to be RDR6-dependent (Howell et al. 2007). Klevebring et al. (2009) described phasiRNAs associated with a small number of NB-LRRs in poplars, but the triggers were not identified. Our cross-species analysis of the pNL-triggering miRNAs demonstrated that at least the miR2118 family (including miR472, miR482, and miR2089) is well conserved in many plant species. miR2118 is particularly interesting because it also triggers phasiRNAs from intergenic regions in rice panicles (Johnson et al. 2009); these are noncoding loci that have no apparent relationship to the miR2118 pNL targets in legumes that we have described.

pNLs potentially function to coregulate en masse the NB-LRR family in legumes. Thus, the small set of pNL triggers could function as master regulators of genes that are the first line of plant defense against many pathogens. The extent of this system in legumes makes it tempting to speculate that this is a critical regulatory circuit that is important for symbiosis. There are data to support this idea; for example, overexpression of the 22-nt miR482 (a member of the miR2118 family) leads to hypernodulation in soybeans (Li et al. 2010). Li et al. (2010) also demonstrated that miR482 is up-regulated 6 d after Bradyrhizobium japonicum inoculation, and the pNL trigger miR1507 is up-regulated in a hypernodulating soybean mutant. Differences in the utilization of pNLs between M. truncatula and soybeans could potentially reflect well-described differences in their nodulation processes. However, the finding that some of these pNL targeting miRNAs are conserved outside of legumes suggests hypotheses other than a role specific to nodulation; one related possibility would be in the global regulation of NB-LRR genes to promote mycorrhizal colonization, a symbiotic interaction in which Arabidopsis does not participate but that is common among legumes and nonleguminous plants and is particularly well studied in potatoes (Hata et al. 2010). This pNL mechanism could have been refined in legumes for rhizobial symbiosis.

Howell et al. (2007) described the role of phasiRNAs in a gene family (PPR-P) that shares some features with the NB-LRR gene family: It is large and dynamic, targeted by more than one miRNA, and may benefit evolutionarily from diversity in the gene family. Howell et al. (2007) suggest that the phasiRNAs could minimize the number of active PPR copies to suppress gene dosage. pNLs may be part of a similar regulatory system to constrain the number of active NB-LRRs. It has been known for many years that the TNL class of NB-LRRs is absent from grass genomes but is found in lower plants (Meyers et al. 1999). We believe that we have identified a phenomenon distinct from the phasiRNA-generating PPR genes because the pNL siRNAs are generated by direct targeting of large numbers of diverse genes by just a few miRNAs that target several distinct, highly conserved, protein motif-encoding sequences. One enduring question has been how an entire Glade of genes could be lost or driven out of a genome. Curiously, the miR2118 family is conserved as a phasiRNA trigger between grasses and legumes, but in legumes it targets NB-LRRs and in grasses it targets noncoding RNAs found in intergenic regions that form clusters (Johnson et al. 2009). Perhaps the reason for the absence of the TNL class from grass genomes is because of phasiRNA suppression of the gene family followed by pseudogenization and, ultimately, neofunctionalization as regulatory noncoding RNAs in grasses, although no function is yet known for the grass phasiRNAs. Extending this hypothesis, perhaps the extensive set of noncoding phasiRNA precursors found in grass genomes is derived from the protein-coding TNL family as pseudogenized, extinct PHAS loci that are vestiges of the extant clusters of pNLs observed in the potato genome, for example (Zhai 2011 Supplemental Fig. S7), which resembles the clustered distribution of those noncoding phasiRNAs loci in grass; such a scenario has been described for the Xist noncoding RNA that functions in X-chromosome inactivation in animals (Duret et al. 2006).

### Diverse Pathways in Legumes are Subject to Regulation by Phased Small RNAs

In addition to the pNL genes, we identified a number of protein-coding genes and miRNA triggers that generate phasiRNAs (Table 2). Among the more unusual is an AP2-like gene with both a miR156 and a conserved miR172 site; miR156 and miR172 are intimately involved in juvenile-to-adult-phase transition in Arabidopsis (Wang et al. 2009; Wu et al. 2009). However, in Arabidopsis, miR156 targets the SPL family upstream of the miR172-targeted AP2-like genes, and the levels of miR156 and miR172 are inversely proportional during maturation of the plant (Wu et al. 2009). The co-occurrence of these miRNA target sites in an AP2-like gene suggests a model in which the AP2 phasiRNAs functions in cis to positively regulate silencing of the AP2 gene (as casiRNAs), while also functioning in trans to target other AP2-like genes.

We also characterized phasiRNA biogenesis from genes important for small

RNA biogenesis and pathogen defense. GmSGS3 is targeted by miR2118, the same miRNAs that target many NB-LRR genes. SGS3 has an important role in juvenile development (Peragine et al. 2004) and is critical in tasiRNA production (Elmayan et al. 2009). The silencing of both NB-LRR genes and SGS3 by miR2118 suggests a coupling of these regulatory events. DCL2 genes in both M. truncatula and soybeans independently acquired 22-nt miRNA target sites, resulting in phasiRNAs from this gene in both species (Zhai 2011 Supplemental Fig. S3A); interestingly, overexpression of miR1515, the soybean DCL2 trigger, was demonstrated to lead to hypernodulation (Li et al. 2010). Target analysis of the M. truncatula DCL2 suggests that the phasiRNA trigger is miR1507, the CNL-specific pNL trigger. Because DCL2 is important for small RNA biogenesis and SGS3 is important for tasiRNA biogenesis, these results are reminiscent of the DCL1-miR162 (Xie et al. 2003) or the AGO1-miR168 feedback loops (Vaucheret et al. 2004, 2006). Since DCL2 and SGS3 are involved in both small RNA silencing and viral resistance (Mourrain et al. 2000), and we demonstrated a substantial network of miRNAs and resulting phasiRNAs that target NB-LRR genes, we propose that the suppression of the small RNA silencing system and disease resistance machinery may play a role in plant-microbe interactions. Therefore, our data are indicative of an extensive regulatory network controlling the transcript levels in these interactions. While most of these findings were made in M. truncatula, we have evidence indicating that parallels exist in other legumes and nonleguminous species, suggesting a small number of miRNAs function as master regulators of the largest gene families found in plant genomes.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

### REFERENCES

Addo-Quaye, C., Miller, W., and Axtell, M. J. (2009). CleaveLand: a pipeline for using degradome data to find cleaved small RNA targets. Bioinformatics 25, 130-131.
Allen, E., and Howell, M. D. (2010). miRNAs in the biogenesis of trans-acting siRNAs in higher plants. Semin Cell Dev Biol 21, 798-804.
Allen, E., Xie, Z., Gustafson, A. M., and Carrington, J. C. (2005). microRNA-directed phasing during trans-acting siRNA biogenesis in plants. Cell 121, 207-221.
Aukerman, M. J., and Sakai, H. (2003). Regulation of flowering time and floral organ identity by a MicroRNA and its APETALA2-like target genes. Plant Cell 15, 2730-2741.
Axtell, M. J., Jan, C., Rajagopalan, R., and Bartel, D. P. (2006). A Two-Hit Trigger for siRNA Biogenesis in Plants. Cell 127, 565-577.
Bartel, D. P. (2004). MicroRNAs: genomics, biogenesis, mechanism, and function. Cell 116, 281-297.
Benedito, V. A., Torres-Jerez, I., Murray, J. D., Andriankaja, A., Allen, S., Kakar, K., Wandrey, M., Verdier, J., Zuber, H., Ott, T., et al. (2008). A gene expression atlas of the model legume Medicago truncatula. Plant J 55, 504-513.
Catalano C M, Lane W S, Sherrier D J 2004. Biochemical characterization of symbiosome membrane proteins from Medicago truncatula root nodules. Electrophoresis 25: 519-531.
Chen, H. M., Chen, L. T., Patel, K., Li, Y. H., Baulcombe, D. C., and Wu, S. H. (2010b). From the Cover: 22-nucleotide RNAs trigger secondary siRNA biogenesis in plants. Proc Natl Acad Sci USA 107, 15269-15274.
Chitwood, D. H., Nogueira, F. T., Howell, M. D., Montgomery, T. A., Carrington, J. C., and Timmermans, M. C. (2009). Pattern formation via small RNA mobility. Genes Dev 23, 549-554.
Cuperus, J. T., Carbonell, A., Fahlgren, N., Garcia-Ruiz, H., Burke, R. T., Takeda, A., Sullivan, C. M., Gilbert, S. D., Montgomery, T. A., and Carrington, J. C. (2010). Unique functionality of 22-nt miRNAs in triggering RDR6-dependent siRNA biogenesis from target transcripts in Arabidopsis. Nat Struct Mol Biol 17, 997-1003.
De Paoli, E., Dorantes-Acosta, A., Zhai, J., Accerbi, M., Jeong, D. H., Park, S., Meyers, B. C., Jorgensen, R. A., and Green, P. J. (2009). Distinct extremely abundant siRNAs associated with cosuppression in petunia. RNA 15, 1965-1970.
Deakin W J, Broughton W J 2009. Symbiotic use of pathogenic strategies: Rhizobial protein secretion systems. Nat Rev Microbiol 7: 312-320.
Devers, E. A., Branscheid, A., May, P., and Krajinski, F. (2011). Stars and symbiosis: microRNA- and microRNA*-mediated transcript cleavage involved in arbuscular mycorrhizal symbiosis. Plant Physiol In Press, doi: 10.1104/pp. 111.172627.
Duret, L., Chureau, C., Samain, S., Weissenbach, J., and Avner, P. (2006). The Xist RNA gene evolved in eutherians by pseudogenization of a protein-coding gene. Science 312, 1653-1655.
Eitas T K, Dangl J L 2010. N B-LRR proteins: Pairs, pieces, perception, partners, and pathways. Curr Opin Plant Biol 13: 472-477.
Elmayan, T., Adenot, X., Gissot, L., Lauressergues, D., Gy, I., and Vaucheret, H. (2009). A neomorphic sgs3 allele stabilizing miRNA cleavage products reveals that SGS3 acts as a homodimer. FEBS J 276, 835-844.
Felippes, F. F., and Weigel, D. (2009). Triggering the formation of tasiRNAs in Arabidopsis thaliana: the role of microRNA miR173. EMBO Rep 10, 264-270.
Franklin, A. E., McElver, J., Sunjevaric, I., Rothstein, R., Bowen, B., and Cande, W. Z. (1999). Three-dimensional microscopy of the Rad51 recombination protein during meiotic prophase. Plant Cell 11, 809-824.
Gebhardt, C., and Valkonen, J. P. (2001). Organization of genes controlling disease resistance in the potato genome. Annu Rev Phytopathol 39, 79-102.
German, M. A., Pillay, M., Jeong, D. H., Hetawal, A., Luo, S., Janardhanan, P., Kannan, V., Rymarquis, L. A., Nobuta, K., German, R., et al. (2008). Global identification of microRNA-target RNA pairs by parallel analysis of RNA ends, Nat Biotechnol 26, 941-946.
Hata, S., Kobae, Y., and Banba, M. (2010). Interactions between Plants and Arbuscular Mycorrhizal Fungi. International Review of Cell and Molecular Biology, Vol 281 281, 1-48.
Howell, M. D., Fahlgren, N., Chapman, E. J., Cumbie, J. S., Sullivan, C. M., Givan, S. A., Kasschau, K. D., and Carrington, J. C. (2007). Genome-wide analysis of the RNA-DEPENDENT RNA POLYMERASE6/DICER-LIKE4 pathway in Arabidopsis reveals dependency on miRNA- and tasiRNA-directed targeting. Plant Cell 19, 926-942.
Jagadeeswaran, G., Zheng, Y., Li, Y. F., Shukla, L. I., Matts, J., Hoyt, P., Macmil, S. L., Wiley, G. B., Roe, B. A., Zhang, W., et al. (2009). Cloning and characterization of small RNAs from Medicago truncatula reveals four novel legume-specific microRNA families. New Phytol 184, 85-98.
Johnson, C., Kasprzewska, A., Tennessen, K., Fernandes, J., Nan, G. L., Walbot, V., Sundaresan, V., Vance, V., and Bowman, L. H. (2009). Clusters and superclusters of phased small RNAs in the developing inflorescence of rice. Genome Res 19, 1429-1440.
Jones-Rhoades, M. W., Bartel, D. P., and Bartel, B. (2006). MicroRNAS and their regulatory roles in plants. Annu Rev Plant Biol 57, 19-53.
Joshi T, Yan Z, Libault M, Jeong D H, Park S, Green P J, Sherrier D J, Farmer A, May G, Meyers B C, et al. 2010. Prediction of novel miRNAs and associated target genes in Glycine max. BMC Bioinformatics 11: S14 doi: 10.1186/1471-2105-11-S1-S14.
Klevebring, D., Street, N. R., Fahlgren, N., Kasschau, K. D., Carrington, J. C., Lundeberg, J., and Jansson, S. (2009). Genome-wide profiling of populus small RNAs. BMC Genomics 10, 620.
Kurtz S, Phillippy A, Delcher A L, Smoot M, Shumway M, Antonescu C, Salzberg S L 2004. Versatile and open software for comparing large genomes. Genome Biol 5: R12 doi: 10.1186/gb-2004-5-2-r12.
Li, H., Deng, Y., Wu, T., Subramanian, S., and Yu, O. (2010). Misexpression of miR482, miR1512, and miR1515 increases soybean nodulation. Plant Physiol 153, 1759-1770.
Liu 3, Maldonado-Mendoza I, Lopez-Meyer M, Cheung F, Town C D, Harrison M J 2007. Arbuscular mycorrhizal symbiosis is accompanied by local and systemic alterations in gene expression and an increase in disease resistance in the shoots. Plant J 50: 529-544.
Lu, C., Jeong, D. H., Kulkarni, K., Pillay, M., Nobuta, K., German, R., Thatcher, S. R., Maher, C., Zhang, L., Ware, D., et al. (2008). Genome-wide analysis for discovery of rice microRNAs reveals natural antisense microRNAs (nat-miRNAs). Proc Natl Acad Sci USA 105, 4951-4956.
Lu, C., Kulkarni, K., Souret, F. F., MuthuValliappan, R., Tej, S. S., Poethig, R. S., Henderson, I. R., Jacobsen, S. E., Wang, W., Green, P. J., et al. (2006). MicroRNAs and other small RNAs enriched in the Arabidopsis RNA-dependent RNA polymerase-2 mutant. Genome Res 16, 1276-1288.
Lu, C., Meyers, B. C., and Green, P. J. (2007a). Construction of small RNA cDNA libraries for deep sequencing. Methods 43, 110-117.
Lu, S., Sun, Y. H., Amerson, H., and Chiang, V. L. (2007b). MicroRNAs in loblolly pine (Pinus taeda L.) and their association with fusiform rust gall development. Plant J 51, 1077-1098.
Mallory, A. C., and Vaucheret, H. (2006). Functions of microRNAs and related small RNAs in plants. Nat Genet 38 Suppl, S31-36.
Meade H M, Long S R, Ruvkun G B, Brown S E, Ausubel F M 1982. Physical and genetic characterization of symbiotic and auxotrophic mutants of Rhizobium meliloti induced by transposon Tn5 mutagenesis. 3 Bacteriol 149: 114-122.
Meyers B C, Kaushik S, Nandety R S 2005. Evolving disease resistance genes. Curr Opin Plant Biol 8: 129-134.
Meyers, B. C., Dickerman, A. W., Michelmore, R. W., Sivaramakrishnan, S., Sobral, B. W., and Young, N. D. (1999). Plant disease resistance genes encode members of an ancient and diverse protein family within the nucleotide-binding superfamily. Plant J 20, 317-332.
Mi, S., Cai, T., Hu, Y., Chen, Y., Hodges, E., Ni, F., Wu, L., Li, S., Zhou, H., Long, C., et al. (2008). Sorting of small RNAs into Arabidopsis argonaute complexes is directed by the 5′ terminal nucleotide. Cell 133, 116-127.
Michelmore R 1996. Flood warning-resistance genes unleashed. Nat Genet 14: 376-378.
Mohorianu, I., Schwach, F., Jing, R., Lopez-Gomollon, S., Moxon, S., Szittya, G., Sorefan, K., Moulton, V., and Dalmay, T. (2011). Profiling of short RNAs during fleshy fruit development reveals stage-specific sRNAome expression patterns. Plant J 67, 232-246.
Montgomery, T. A., Howell, M. D., Cuperus, J. T., Li, D., Hansen, J. E., Alexander, A. L., Chapman, E. J., Fahlgren, N., Allen, E., and Carrington, J. C. (2008a). Specificity of ARGONAUTE7-miR390 interaction and dual functionality in TAS3 trans-acting siRNA formation. Cell 133, 128-141.
Montgomery, T. A., Yoo, S. J., Fahlgren, N., Gilbert, S. D., Howell, M. D., Sullivan, C. M., Alexander, A., Nguyen, G., Allen, E., Ahn, J. H., et al. (2008b). AGO1-miR173 complex initiates phased siRNA formation in plants. Proc Natl Acad Sci USA 105, 20055-20062.
Mourrain, P., Beclin, C., Elmayan, T., Feuerbach, F., Godon, C., Morel, J. B., Jouette, D., Lacombe, A. M., Nikic, S., Picault, N., et al. (2000). Arabidopsis SGS2 and SGS3 genes are required for posttranscriptional gene silencing and natural virus resistance. Cell 101, 533-542.
Pawlowski, W. P., Golubovskaya, I. N., and Cande, W. Z. (2003). Altered nuclear distribution of recombination protein RAD51 in maize mutants suggests the involvement of RAD51 in meiotic homology recognition. Plant Cell 15, 1807-1816.
Pawlowski, W. P., Golubovskaya, I. N., Timofejeva, L., Meeley, R. B., Sheridan, W. F., and Cande, W. Z. (2004). Coordination of meiotic recombination, pairing, and synapsis by PHS1. Science 303, 89-92.
Peragine, A., Yoshikawa, M., Wu, G., Albrecht, H. L., and Poethig, R. S. (2004). SGS3 and SGS2/SDE1/RDR6 are required for juvenile development and the production of trans-acting siRNAs in Arabidopsis. Genes Dev 18, 2368-2379.
Schmutz J, Cannon S B, Schlueter J, Ma J, Mitros T, Nelson W, Hyten D L, Song Q, Thelen J J, Cheng J, et al. 2010. Genome sequence of the palaeopolyploid soybean. Nature 463: 178-183.
Soderlund C, Bomhoff M, Nelson W M 2011. SyMAP v3. 4: A turnkey synteny system with application to plant genomes. Nucleic Acids Res 39: e68.
Song, Q. X., Liu, Y. F., Hu, X. Y., Zhang, W. K., Ma, B., Chen, S. Y., and Zhang, J. S. (2011). Identification of miRNAs and their target genes in developing soybean seeds by deep sequencing. BMC Plant Biol 11:5.
The_International_Brachypodium_Initiative (2010). Genome sequencing and analysis of the model grass Brachypodium distachyon. Nature 463, 763-768.
Valdes-Lopez O, Yang S S, Aparicio-Fabre R, Graham P H, Reyes J L, Vance C P, Hernandez G 2010. MicroRNA expression profile in common bean (Phaseolus vulgaris) under nutrient deficiency stresses and manganese toxicity. New Phytol 187: 805-818.
VandenBosch K A, Rodgers L R, Sherrier D J, Kishinevsky B D 1994. A peanut nodule lectin in infected cells and in vacuoles and the extracellular matrix of nodule parenchyma. Plant Physiol 104: 327-337.
Vaucheret, H. (2006). Post-transcriptional small RNA pathways in plants: mechanisms and regulations. Genes Dev 20, 759-771.
Vaucheret, H., Mallory, A. C., and Bartel, D. P. (2006). AGO1 homeostasis entails coexpression of MIR168 and AGO1 and preferential stabilization of miR168 by AGO1. Mol Cell 22, 129-136.
Vaucheret, H., Vazquez, F., Crete, P., and Bartel, D. P. (2004). The action of ARGONAUTE1 in the miRNA pathway and its regulation by the miRNA pathway are crucial for plant development. Genes Dev 18, 1187-1197.
Wang, J. W., Czech, B., and Weigel, D. (2009). miR156-regulated SPL transcription factors define an endogenous flowering pathway in Arabidopsis thaliana. Cell 138, 738-749.
Wu, G., Park, M. Y., Conway, S. R., Wang, J. W., Weigel, D., and Poethig, R. S. (2009). The sequential action of miR156 and miR172 regulates developmental timing in Arabidopsis. Cell 138, 750-759.
Xie, Z., Kasschau, K. D., and Carrington, J. C. (2003). Negative feedback regulation of Dicer-Like1 in Arabidopsis by microRNA-guided mRNA degradation. Curr Biol 13, 784-789
Xu, X., Pan, S., Cheng, S., Zhang, B., Mu, D., Ni, P., Zhang, G., Yang, S. P. I., Li, R. P. I., Wang, J. P. I., et al. (2011). Genome sequence and analysis of the tuber crop potato. Nature 475, 189-195.
Yang, S., Tang, F., Gao, M., Krishnan, H. B., Zhu, H. 2010. R gene-controlled host specificity in the legume-rhizobia symbiosis. Proc Natl Acad Sci 107: 18735-18740.
Yoshikawa, M., Peragine, A., Park, M. Y., and Poethig, R. S. (2005). A pathway for the biogenesis of trans-acting siRNAs in Arabidopsis. Genes Dev 19, 2164-2175.
Zhai, 1, Jeong, D., Paoli, E. D., Park, S., Rosen, B. D., Li, Y., Gonzalez, A. J., Yan, Z., Kitto, S. L., Grusak M. A., Jackson S A, Stacey G, Cook D R, Green P J, Sherrier D J, Mayers B C. (2011). MicroRNAs as master regulators of the plant IVB-LRR defense gene family via the production of phased, trans-acting siRNAs. Genes & Development 25:2540-53.

TABLE 1
miRNA Sequences

| SEQ | |
|---|---|
| ID | |
| NO | Sequence |
| 1 | UGAACAGAAAGAACAUUUGGC |
| 2 | UAGGGACUAAAUUGAUGGUUU |
| 3 | AUUCAAGAAGGUCGUGGAAAA |
| 4 | CCACAUCAAUGGUCGUUGGAU |
| 5 | UGAGAACUGUUUUCCGCACCUU |
| 6 | UAGGUAUUUUAAGGAGCACGUU |
| 7 | CGUGACAACACAUCAUUGGAUGCA |
| 8 | UGGAUUCAACUCUAAGUGUCGGUU |
| 9 | AAAAGAAUACUCAUACAUAACAUU |
| 10 | ACAAAGACAUUGGAAGGCUUA |
| 11 | UUCGGGUUGAUAAUAUUUUCUU |
| 12 | UGUGAAUGAUGCGGGAGCUAA |
| 13 | UAAAUGUGUUGGAAUUAAGGUU |
| 14 | UGAUAGAGCUAGACCAUCGGAG |
| 15 | AAGAGAGAUAGAUCAGAAUUGACA |
| 16 | UUGAUUUGAUCAGAUGGUUUU |
| 17 | UUGAUCAAGGACUUUGCAUC |
| 18 | CAUUGUUUGGUUUAGUACAAA |
| 19 | AUUUUGAUUGGUGCCAACUAA |
| 20 | UAUUGCACUCAUCUUCCAUGGC |
| 21 | CUUUAACGCGGGAAAGACACA |
| 22 | UGAUGACGGAAGAAAUCCAAA |
| 23 | UCUGUCAGUAGACUCAAUUUC |
| 24 | UGAAAGUGUAGAGAGUAGAUU |
| 25 | UGAAAGUGUGGAGAGUAGAUU |
| 26 | UUAGCAGGAAGAGUGACUAUG |
| 27 | UUUUCCAAUUCUAAGUCUAUC |
| 28 | CCGACCGGAUUCUCAGACGGG |
| 29 | UAGAGAUUUGUUUAACAGCCA |
| 30 | UGAGAAUGUUAGAUACGGAAC |
| 31 | UACUUGGUGAAUUGUUGGAUC |
| 32 | UAAGUUGGAAGAAUGUAUUUG |
| 33 | UUAUGCAAGUAGAUAAGCUCA |
| 34 | UUUAAUUUAUAUACAUCGUCA |
| 35 | AAGGGGGUGAAAAGAUUCAAA |
| 36 | AUACAUCAUUGAUUGAAUGAACCU |
| 37 | UGCUUUAAGGAUAUUUGUUAAGGA |
| 38 | AUGGGAUCCUGUUGGUGGGUUAC |
| 39 | AACCCUCAAAGGCUUCCACGG |
| 40 | GUGGGAUCCGUUGAUGGGUUAC |
| 41 | CAUUUGGAGAGACAUAGACAA |
| 42 | UGGGAGAAAAGAUAGAAUGUG |
| 43 | GGAGAUGGGAGGGUCGGUAAAG |
| 44 | UUUUGUGUCGUGAAGCUUUUG |
| 45 | CAUGGAAGUGAAUCGGGUGAC |
| 46 | UCAUAGGAGGAAUCAACUGGC |
| 47 | UGAACUAUACAAAGACGGUUA |
| 48 | UCCAUUCGCGGACAUGAUGGAU |
| 49 | CGAAACGUUGAGGUAUAUGUGGAC |
| 50 | UUGAGGCUGAGAAGAGGCAAG |
| 51 | UGGAGGACCUUUGAAGGUGCA |
| 52 | AAGUGCAAUACUGAUCUUCGGAAC |
| 53 | UGAGGGAAAUGAAGACGACGA |
| 54 | UUAGGACUAUGGUUUGGACGA |
| 55 | CCUGUCGUAGGAGAGAUGACGC |
| 56 | UUUGGGAUCAUGCUAUCCCUU |
| 57 | AUCUCAAGUGGAUUGCUUAAGGAC |
| 58 | CUGGGUGAGAGAAACACGUAU |
| 59 | AGAAUGUGAGUUAGAGUGAGCAUC |
| 60 | UUUUUAAAAGGUUCAGUUAGGU |
| 61 | GCUGGCGUCGACACGUGGCAU |
| 62 | UGGGUGAGAGAAACGCGUAUC |
| 63 | AAUGUCUGGGCUUAGCGAGGCGGU |
| 64 | AUAAGCUCUUUUGAGAGCUUC |
| 65 | AAAGCCAUGACUUACACACGC |
| 66 | UUAUAGUCUGACAUCUGGAAU |
| 67 | AACUUGGGCUGAGCUUAGGUG |
| 68 | CUAGCAAUAAUGUUGGAUGCAC |
| 69 | CGACGAACUCUUCGUCGGCAUC |
| 70 | CAAGUCCAAAGUAGGAAUGUUGCA |
| 71 | AUCACUUAGCUGACGGUAGGGAC |
| 72 | CUGAAACUGAGACUGCAUCUGG |
| 73 | UAGCUGGUAGGAGAAGUUCAG |
| 74 | GACGACGACGGGGAGGACGCGC |
| 75 | AAACAGAUCUAAAUGGAUUCC |
| 76 | UUAGGACUAUGGUUUGGACAA |
| 77 | AAUUAGCUAAUGGUUAGCUAA |
| 78 | UAGUGUUGUCCUGUCGAACACGGA |
| 79 | AGUGGAACUUUGAGGCCUGC |
| 80 | UGAGAAUUUGGCCUCUGUCCA |
| 81 | UAAUUGUGUUGUACAUUAUCA |
| 82 | UUGACAGAAGAGGGUGAGCACA |
| 83 | ACAGGGAACAUGCAGAGCAUG |
| 84 | AGCUGCUGACUCGUUGGUUCA |
| 85 | GGCAGGUCAUCCUUCGGCUAUA |
| 86 | UACGUGUGUCUUCACCUCUGAA |
| 87 | CGAGCCGAAUCAAUAUCACUC |
| 88 | UCUCGGACCAGGCUUCAUUCC |
| 89 | UUGAGCCGCGCCAAUAUCACU |
| 90 | GCUCAAGAAAGCUGUGGGAGA |
| 91 | UGAGCCAAAGAUGACUUGCCGG |
| 92 | UCGCUUGGUGCAGGUCGGGAA |
| 93 | AUCAUGCUAUCCCUUUGGAUU |
| 94 | GGAGUGACACUGAGAACACAAG |
| 95 | UCGCAGGAGAGAUGACACCUUC |
| 96 | UUUGGUACUUCGUCGAUUUGAU |
| 97 | UUAAUCUAGGAAAAUACGGUGG |
| 98 | UGAAGCUGCCAGCAUGAUCUG |
| 99 | AUCCUUGGAAUGCAGAUUAUC |
| 100 | UUUGGCAUUCUGUCCACCUCC |
| 101 | AGAAUCUUGAUGAUGCUGCAU |
| 102 | AUUGGAGUGAAGGGAGCUCCA |
| 103 | UGCAUUUGCACCUGCACUUUC |
| 104 | UGCCAAAGGAGAGUUGCCCUG |
| 105 | CAGGGGAACAGGCAGAGCAUG |
| 106 | GCGUAUGAGGAGCCAAGCAUA |
| 107 | UAUGGGGGGAUUGGGAAGGAA |
| 108 | UUGGACUGAAGGGAGCUCCCU |
| 109 | UCUUGCUCAAAUGAGUAUUCCA |
| 110 | UUGACAGAAGAAAGGGAGCAC |
| 111 | UGCAUUUGCACCUGCACUUUA |
| 112 | UUGAGCCGCGUCAAUAUCUCA |
| 113 | UGAAUCUUGAUGAUGCUGCAU |
| 114 | UGACAGAAGAGAGUGAGCACU |
| 115 | UUGAGCCGCGCCAAUAUCACU |
| 116 | GCUCAAGAAAGCUGUGGGAGA |
| 117 | UUGAGCCGUGCCAAUAUCACA |
| 118 | UACGCAGGAGAGAUGACGCUG |
| 119 | GGAAUCUUGAUGAUGCUGCAU |
| 120 | UGAGCCGGGAUGGCUUGCCGGCA |
| 121 | UGAGCCAGGAUGGCUUGCCGGC |
| 122 | UUUGGACCGAAGGGAGCCCCU |
| 123 | UAUUGACGCUGCACUCAAUCA |
| 124 | CGGGCAAGUUGUUUUUGGCUAC |
| 125 | AGGUGGGCAUACUGUCAACUG |
| 126 | UUGGCAUUCUGUCCACCUCC |
| 127 | UGAGACCAAAUGAGCAGCUGA |
| 128 | UAGAAGCUCCCCAUGUUCUCA |
| 129 | UGAGCCAAGGAUGACUUGCCG |
| 130 | UCUCGGACCAGGCUUCAUUCC |
| 131 | CUGACAGAAGAUAGAGAGCAU |
| 132 | AGGGAUAGGUAAAACAAUGAC |
| 133 | UAGAAAGGGAAAUAGCAGUUG |
| 134 | UGAUUGAGCCGUGCCAAUAUC |
| 135 | AUUCAAGAUAGCUGUGGAAAA |
| 136 | UCGAUAAACCUCUGCAUCCAG |
| 137 | AGCUCUGUUGGCUACACUUUG |
| 138 | AAGCCAAGGAUGACUUGCCGG |
| 139 | AACCAGGCUCUGAUACCAUGG |
| 140 | AAGUUGUGAUGAGAAUCAAUGGCA |
| 141 | UAAUCUGCAUCCUGAGGUUUA |
| 142 | CUGACAGAAGAUAGAGAGCAC |
| 143 | UGUCGCAGGAUAGAGGGCACU |
| 144 | AUGCACUGCCUCUUCCCUGGC |
| 145 | GUCCUUGGGAUGCAGAUUACG |
| 146 | AAAAGCACUUAAGGAACGGUA |
| 147 | AGGGAUAGGUAAAACAACUAC |
| 148 | GGAAUGGGCUGAUUGGGAAGC |
| 149 | UAACUGAACAUUCUUAGAGCAU |
| 150 | UAACUGGAAAUUCUUAAAGCAU |
| 151 | UAGCCAAGAAUGACUUGCCGG |
| 152 | CUGGGAACAGGCAGGGCACGA |
| 153 | CGAGCCGAAUCAAUACCACUC |
| 154 | UGCGAGUGUCUUCGCCUCUGA |
| 155 | UGCGGGUAUCUUUGCCUCUGA |
| 156 | UUAAUCAAGGAAAUCACGGUUG |
| 157 | UGCCAAAGGAGAUUUGCCCAG |
| 158 | UAGCCAAGGAUGGACUUGCCUA |
| 159 | GGAAUGUUGUCUGGCUCGAGG |
| 160 | CUUCCAUAUCUGGGGAGCUUC |
| 161 | AUUGAGUGCAGCGUUGAUGAA |
| 162 | UGUGUUCUCAGGUCGCCCCUG |
| 163 | UGAAGUGUUUGGGGGAACUUU |
| 164 | CGGACCAGGCUUCAUUCCCC |
| 165 | AGAGGUGUUUGGGAUGAGAGA |
| 166 | CAGCCAAGGAUGACUUGCCGU |

TABLE 2
M. truncatula PHAS loci triggered by legume miRNAs

| | Length | | Number of PHAS |
|---|---|---|---|
| miRNA | (nt) | Targeted Gene Family | loci in Medicago |
| miR390 | 21 | TAS3 | 1 |
| miR172 | 21 | AP2-like | 1 |
| NEW-m1059 | 22 | Predicted protein kinase | 1 |
| miR2109 | 22 | NB-LRRs | 25 |
| miR2118a/b/c | 22 | NB-LRRs, Gma-SGS3, etc. | 32 |
| miR1507 | 22 | NB-LRRs, Mtr-DCL2 | 14 |
| miR1509 | 22 | Predicted | 4 |
| | | transcription factor | |
| miR1515 | 22 | Gma-DCL2 | — |

1. A method for generating a phased small RNA (phasiRNA) in a plant cell, comprising introducing into the plant cell an effective amount of a microRNA (miRNA).

2. The method of claim 1, wherein the miRNA is 22-nt in length.

3. The method of claim 1, wherein the miRNA is selected from the group consisting of miR156, miR161, miR162, miR167, miR168, miR169, miR172, miR173, miR389*, miR390, miR393, miR472, miR482, miR772, miR828, miR1507, miR1509, miR1510, miR1512, miR1515, miR2089, miR2109, miR2118a, miR2118b, miR2118c, miR2597, miR5300, and miR5754.

4. The method of claim 1, wherein the miRNA is selected from the group consisting of miR1507 family, miR2109 family and miR2118 family.

5. The method of claim 4, wherein the miR1507 family comprises gma-miR1507a, mtr-miR1507, gma-miR1507b, vun-miR1507a, vun-miR1507b, gso-miR1507a, gso-miR1507b, gma-miR1507c.

6. The method of claim 4, wherein the miR2109 family comprises gma-miR2109, gso-miR2109.

7. The method of claim 4, wherein the miR2118 family comprises pvu-miR2118, mtr-miR2118, osa-miR2118a, osa-miR2118b, osa-miR2118c, osa-miR2118d, osa-miR2118e, osa-miR2118f, osa-miR2118g, osa-miR2118h, osa-miR2118i, osa-miR2118j, osa-miR2118k, osa-miR2118l, osa-miR2118m, osa-miR2118n, osa-miR2118o, osa-miR2118p, osa-miR2118q, osa-miR2118r, zma-miR2118a, zma-miR2118b, zma-miR2118c, zma-miR2118d, zma-miR2118e, zma-miR2118f, zma-miR2118g, gma-miR2118a, gma-miR2118b, vun-miR2118.

8. The method of claim 1, wherein the miRNA is selected from the group consisting of miR472, miR482, miR2089, miR2118a, miR2118b, and miR2118c.

9. The method of claim 1, wherein the miRNA has a sequence selected from the group consisting of SEQ ID NO: 1-166.

10. The method of claim 1, whereby a transcript of a NB-LRR encoding gene in the plant cell is cleaved.

11. The method of claim 10, wherein the NB-LRR encoding gene is a disease resistance gene.

12. The method of claim 1, wherein the phasiRNA is generated from a gene selected from group consisting of DCL2 and 5G53.

13. The method of claim 1, wherein the plant cell is in a legume.

14. The method of claim 13, wherein the legume is selected from the group consisting of M. truncatula, soybeans, peanuts, and common.

15. The method of claim 1, wherein the plant cell is in a non-legume.

16. The method of claim 15, wherein the non-legume is selected from the group consisting of corns, rice, wheat, barley, oats, rye, sorghum, sugar cane, grapevine, almonds, apple, peach, sugar beet, tomato, potato, tobacco, cotton, lettuce, sunflower, melons, strawberries, and canola.

17. The method of claim 13, wherein resistance of the plant to a microbe is reduced.

18. The method of claim 13, wherein the plant enters a symbiotic interaction with a microbe.

19. The method of claim 17, wherein the microbe is a rhizobial strain.

20. The method of claim 13, wherein nodulation in the plant is improved.
